(19) United States
(12) Patent Application Publication
(10) Pub. No.: US 2025/0066840 A1
(43) Pub. Date: Feb. 27, 2025

(54) Single-cell RNA sequencing analysis pipeline for predicting cell sensitivities to various drugs

(71) Applicant: Regents of the University of Minnesota, Minneapolis, MN (US)

(72) Inventors: R. Stephanie Huang, Roseville, MN (US); Weijie Zhang, Columbus, OH (US); Danielle Maeser, Rosemount, MN (US); Yingbo Huang, St. Paul, MN (US)

(21) Appl. No.: 18/802,941

(22) Filed: Aug. 13, 2024

Related U.S. Application Data
(60) Provisional application No. 63/532,973, filed on Aug. 16, 2023.

(51) Int. Cl. C12Q 1/6809; G16B 25/10; G16B 40/20

(52) U.S. Cl. CPC C12Q 1/6809; G16B 25/10; G16B 40/20; C12Q 2600/106

(57) ABSTRACT

Methods for facilitating drug discovery in various models and proposing single cell-type-specific drug candidates, and predicting cellular drug sensitivities. Predictions comprise obtaining bulk RNA sequence data, wherein bulk RNA seq data is pan-cancer cell line transcriptomic data from one or more sources; obtaining single cell RNA sequence data (scRNA-seq) from one or more sources; integrating the bulk RNA sequence data and scRNA-seq data; capturing similarities between the bulk data and the scRNA-seq data based on a shared drug response relative gene and using canonical correlation analysis; extracting one or more drug-response relevant features and selecting a pharmacogenomic subspace for accurately predicting a single-cell drug response.

### BACKGROUND

The present invention relates to methods for predicting drug response scores in single cell RNA-sequence data.

Heterogeneity within tumors, where distinct cell subpopulations display varying phenotypic features, has been causally linked to therapy resistance and disease recurrence in many cancers (Jamal-Hanjani et al., 2015, Schmidt et al., 2016, McGranahan et al, 2017). Tumor cells unresponsive to standard-of-care (SOC) pharmacological interventions continue to proliferate and cascade disease progression under the selective pressure. Such phenotypic aberrations often correlate with molecular variations in cellular mutational and transcriptional profiles (Kim et al., 2015, Liu et al., 2018, Saeed et al, 2019). Thanks to the quickly evolving single-cell (SC) sequencing technologies, genomic and transcriptomic landscapes within tumors in many patient populations have been continuously characterized (Ortega et al., 2017, Wu et al., 2021). On the other hand, the increasingly available single-cell sequencing data confers opportunities for new treatment strategies that tackle problematic cell groups, address clonal heterogeneity, and eventually help achieve curability in cancers (Heath et al, 2016, Van de Sande et al., 2023).

In addition to traditional pharmaceutical research and development pipelines, computational frameworks have emerged as an indispensable tool for drug discovery due to the cost-efficient nature and more importantly, the ability to screen many drugs for various indications (Karaman et al., 2019, Pushpakom et al., 2019, Adam et al., 2020). Current in silico drug discovery models are largely constructed based on openly available high-throughput drug screens on pan-cancer cell lines (CCLs) (Ling et al., 2018, Rees et al., 1986), whose transcriptomic profiles are systematically evaluated through bulk RNA sequencing (RNA-seq) (Barretina et al., 2012). While computational tools utilizing relationships between CCL gene expression from RNA-seq and drug response have demonstrated practicality in predicting efficacious treatments (Adam et al., 2020, Geeleher et al., 2014, Suphavilai et al. 2018, Smith et al., 2020), such relationships cannot be directly applied to generate predictions of drug response at the cellular level, as RNA-seq is limited to measuring average expression across a diverse set of cells, which obscures cell type and composition, as well as temporal and spatial distributions. Thus, inferring cellular drug response requires specialized tools to transfer current drug-gene information to single-cell RNA sequencing (scRNA-seq) data that encapsulate cell level gene expression patterns (Wu et al, 2020, Suphavilai et al, 2021, Fustero-Torre et al, 2021).

In recent years, such computational tools have been conceptualized, and a few implementations have also been proposed (Van de Sande, B., 2023, Wu, Z., 2020, Ji, Y. 2021). The common crucial functionality among the proposed methods relates to overcoming fundamental differences in properties of bulk and SC RNA-seq data to enable predictions of drug response at the SC resolution using learned drug-gene information from bulk data. To achieve this, Beyondcell, DREEP, and scDr choose to learn a fixed number of drug-specific signature genes from bulk CCL data and apply learned signatures independently in scRNA-seq data to calculate signature scores which indicate drug response (Fustero-Torre, C. 2021, Gambardella, G. et al. 2022, Lei, W. 2023). However, considering that genes may harbor varying predictability for response to different drugs and scRNA-seq data are notorious for their low detection rates as well as stochastic drop-outs (Ling, A et al. 2018, Qiu, P. 2020), it is not guaranteed that gene signatures always deliver reliable predictions of sensitivities to various drugs (Qi, X., et al. 2020). In comparison, SCAD and scDEAL directly tackle differences between bulk and SC data and emphasize integration of the two domains via neural network based approaches (Chen, J et al., 2022). While data-hungry deep learning (DL) routes could benefit from large scRNA-seq data and model complex drug-gene relationships, the availability of CCL bulk data could pose continued limits against accurate parameter estimation. Also, it has been shown that DL methods offer comparable performances as classical machine learning frameworks in drug response modeling (Smith, A M et al., 2020), while in general consist of more parameters and request more computing resources. CaDRReS-Sc also conducts bulk-SC data integration but through projecting original data into a fixed-dimensional subspace (Suphavilai C. et al., 2021). These integration-embedded methods by default use dichotomous labels for drug response (sensitive or resistant), and such arbitrary cutoffs often may not reflect pharmacological properties and could mask variation of drug response among heterogeneous cells. Furthermore, insufficient evidence has been presented thus far to demonstrate the translational value of these predictive models in aiding cell-type aware early development for diverse biology models.

Further, assessments of gene expression at the single cell (SC) level offer detailed mappings of cell compositions and substantially advance understandings of complex diseases such as cancer that involves heterogeneous cell types. Origins of therapy resistance and disease recurrence have been linked to such heterogeneity in many malignancies, often attributed to existence of insusceptible cells thriving under selective pressures (Jamal-Hanjani M. et al. 2015, Schmidt, F. et al., 2016, McGranahan N., et al., 2017). Accordingly, cell-type-aware drug discovery using scRNA-seq data has demonstrated potentials to curb resistance and improve treatment outcomes (Sade-Feldman M. et al., 2018, Cohen, Y C. Et al., 2021, Abdelfattah, N. et al., 2022). Though computational drug discovery tools have been proposed for this goal amid increasing public access to scRNA-seq datasets, most pipelines still focus on target identification and validation, a procedure often involves generation of scRNA-seq data, or inference of cellular changes under therapeutic perturbations (instead of cellular response to a potential treatment given its expression profile) (Van de Sande, B. et al., 2023), Lotfollahi M. et al., 2021). These methods so far have not been demonstrated to be able to utilize existing scRNA-seq data to conduct virtual drug screens, facilitate hypothesis formulation, and propose drug candidates addressing tumor heterogeneity.

### SUMMARY

An aspect of the present disclosure relates to methods for predicting a cellular drug response by obtaining single cell RNA sequence data (scRNA-seq); obtaining bulk RNA sequence data, wherein the bulk RNA sequence data is for a cancer cell line and integrating the scRNA-seq data with bulk RNA sequence data and obtaining one or more shared gene expression patterns. Capturing similarities between the scRNA-seq data and the bulk RNA sequence data and selecting one or more features for extracting drug response informative features with applying one or more known drug-gene relationships to the scRNA-seq data allows for predicting or inferring a cellular drug response score.

Re-mapping of the bulk RNA sequence data onto a subspace with significantly lower dimensionality and less noise facilitates downstream predictions.

The method can be scaled to handle large data sets and integrate the scRNA-seq data with multiple drug screen databases for accuracy in predicting drug response scores.

In one or more embodiments, capturing similarities between the scRNA-seq data and the bulk RNA sequence data comprises canonical correlation analysis.

Another aspect of the present disclosure relates to methods for predicting a cellular drug response score from bulk RNA sequence data, wherein bulk RNA seq data is pan-cancer cell line transcriptomic data from one or more sources and single cell RNA sequence data (scRNA-seq) from one or more sources. Integrating the bulk RNA sequence data and scRNA-seq data; capturing similarities between the bulk data and the scRNA-seq data based on a shared drug response relative gene; and extracting one or more drug-response relevant features and selecting a pharmacogenomic subspace allow for accurately predicting a single-cell drug response.

In one or more embodiments, the method includes detecting drug response relevant genes and ranking the drug response relevant genes by an associated adjusted p-value, ranking the drug response relevant genes from most significantly associated with a drug response to least.

In one or more embodiments the method comprises filtering the scRNA-seq data for cells with at least 200 genes detected and genes detected in at least 3 cells and normalizing each cell to have the same total counts of one million counts per million and log-transformed through log 2(CPM+1).

When integrating, the bulk RNA data has a sequence matrix according to X_{bk} ∈ R^{n2xp} with n₁ samples and p genes and the single cell RNA data has a sequence matrix of X_{sc} ∈ R^{n2xp} with n₂ cells and p genes and each of the matrices have a same drug response relative gene. Capturing similarities between the bulk data and the single cell sequence data with a singular value decomposition on the matrix derived is based on the multiplication of X_{bk}X_{sc}^{T}. X_{bk}X_{sc}^{T}.

Using SVD(X_{bk}X_{sc}^{T})=USV^{T}=Σᵢ₌₁^{k} uᵢsᵢvᵢ^{T} as a correlation vector wherein singular vectors U=(u₁, u₂, . . . , uₙ_{₁}) correspond to a correlation vector for the bulk RNA sequence data and wherein singular vectors V=(v₁, v₂, . . . , Vₙ_{₂}) correspond to a correlation vector for the scRNA-seq data allows for capturing similarities.

Extracting comprises correlating each feature in Z_{bk} for the bulk RNA sequence data embeddings with a known drug response.

Adjusting resulting p-values via a Benjamini-Hochberg procedure controls false discovery rates and features that have a false discovery rate less than a selected threshold (S) are retained.

The pharmacogenomic subspace comprises dimensions r={r₁, r₂, . . . , rⱼ}c {1, 2, . . . , k}wherein the threshold is δ ∈ {0.05, 0.1}.

The cellular drug response is predicted according to Xₜₑₛₜ=Z_{sc}^{n2xr}.

Yet another aspect of the present disclosure relates to a method for facilitating drug discovery in various models by proposing cell-type-specific drug candidates by selecting one or more drug response relevant genes; integrating input cancer cell line bulk RNA sequence data and single cell RNA sequence data and preserving shared expression patterns between the bulk RNA sequence data and the single cell RNA sequence data while reducing noise; embedding the bulk RNA sequence data with known cancer cell line drug responses; extracting drug response informative features from the resulting embeddings; constructing one or more regression models; and applying learned coefficients to single cell RNA sequence embeddings to predict cellular drug sensitivity scores.

Integrating comprises using varying single cell sample sizes to drug response relative gene ratios from 10 to less than 1.

Yet another aspect of the present disclosure relates to a framework for learning relationships between drug sensitivities and relevant gene expression patterns based on cancer cell line RNA sequence data and cancer cell line drug screen results comprising integrating a cancer cell line RNA sequence dataset and target single cell RNA sequence dataset to de-noise and extract shared gene expression patterns between the cancer cell line RNA sequence data and single cell RNA sequence data, wherein resulting bulk data is then used to train drug response models and wherein corresponding coefficients are further applied to the single cell RNA sequence data to infer a cellular drug sensitivity score.

Employing canonical correlation analysis captures similarities between cells based on corresponding single cell RNA sequence data and cancer cell line RNA sequence data.

### BRIEF DESCRIPTION OF THE DRAWINGS

FIG. 1 is a schematic overview of scIDUC. Drug response relevant genes (DRGs) were generated prior to the scIDUC pipeline. Step 1 integrates input CCL bulk RNA-seq data and scRNA-seq data to preserve shared expression patterns while reducing noise. Resulting embeddings of bulk RNA-seq were used with CCL drug response to extract drug response informative features and construct regression models in Step 2. Learned coefficients were applied to scRNA-seq embeddings to infer cellular drug sensitivity scores.

FIGS. 2A-2C illustrate scIDUC recapitulates cellular drug sensitivity status in scRNA-seq data. scIDUC applied with or without integration using varying single-cell sample size to DRG ratios from 10 to less than 1. 50 bootstrap samples were generated within each ratio; “all” indicates the sc-DRG ratio when using all available DRGs. Common-language effect Rho and Cohen'D comparing predicted cell response between the true resistant vs. sensitive cell groups across three scRNA-seq datasets. A larger value indicates a higher separation between the groups. FIG. 2A, top row: Lung-PC9; FIG. 2B, mid row: Breast-MCF7; and FIG. 2C bottom row: AML-PDX. Basic information of these dataset can be found in TABLE 1. Summarized numeric results are shown in TABLE 2.

FIGS. 3A and 3B illustrate scIDUC outperforms other methods across three scRNA-seq datasets. For each method, 50 bootstrap samples were used. In all three datasets, scIDUC shows higher Common-language effect Rho (FIG. 3A) and Cohen' D (FIG. 3B) comparing predicted cell response between the true resistant vs. sensitive cell groups than that of other methods (CaDRReS-Sc or Beyondcell). Summarized numeric results are shown in TABLE 3.

FIGS. 4A-4F illustrate scIDUC facilitates drug discovery in various models by proposing cell-type-specific drug candidates. FIG. 4A. Top drug targets predicted by scIDUC for chemo-resistant mesoderm cells in RMS-oPDX (FIG. 4B). Efficacious drugs for mesoderm cells in RMS-oPDX concurrently identified from the CTRPv2 and the GDSC2 and their targets are shown in FIG. 4C. ScIDUC recapitulates differential efficacies shaped by tumor microenvironments in the PDAC model CFPAC1 cells as shown in FIG. 4D. Frequent targets of drugs predicted to be efficacious against docetaxel resistant cells, FIG. 4E. MEK inhibitor selumetinib is predicted to be effective against docetaxel-resistant DU145 cells, FIG. 4F. An in vitro system consisting of docetaxel sensitive and resistant DU145 cells was used to evaluate prediction results.

FIG. 5 illustrates the integration of CCL RNA-seq and single-cell RNA-seq datasets via CCA, obtaining a low-rank approximate of the original matrix X_{bk}X_{sc}^{T}. The left and right singular vectors are considered canonical correction vectors and constitute as the features in the subspace where original bulk and SC datasets are projected onto.

FIGS. 6A-6C illustrate the robustness of CCA and NMF based integration for single-cell drug response prediction.

FIG. 7A (Raghavan et al., 2021) and 7B are charts showing prospective analysis to capture therapeutic vulnerabilities in PDAC-CFPAC1 cells shaped by TMEs. In FIG. 7A, drug panel screens showing differential efficacy in a pancreatic ductal carcinoma patient-derived xenograft model with two different subtypes due to different TMEs are illustrated, this figure was originally generated by Raghavan et al. (FIG. 6G in DOI:https://doi.org/I0.1016/j.cell.2021.11.017). FIG. 7B illustrates predicted cellular response to various drugs using scIDUC. T-tests results comparing predicted nAUCs between scBasal and scClassical cells are shown (right).

FIGS. 8A-8C illustrate the robustness of CCA and NMF based integration for single-cell drug response prediction, implementing scIDUC with different integration methods (CCA or NMF) and drug response models (Lm: linear model; Kernel: kernelized nonparametric regression). Effect size Coden's D comparing predicted nAUC between true resistant and sensitive groups in each data and experiment setting is shown. A Cohen's D>0 indicates correct direction (green); a value smaller than 0 indicates incorrect predicted trends (red).

FIG. 9 illustrates that Docetaxel-resistant DU145 cells show higher resistance to docetaxel compared to their docetaxel-sensitive counterparts in vitro (two-way ANOVA p<0.0001). At each concentration, mean percent viability ±standard deviation is plotted. This is to confirm the establishment of our DU145 docetaxel-resistant model system.

FIG. 10 is a Manhattan plot displaying the significance of associations between each feature in the bulk embeddings with measured gefitinib response. The x-axis represents the index of the features in the bulk embedding, where there are 507 features in total. The y-axis shows the logarithm of the raw p-value from Spearman correlation analysis. The dashed green line indicates the false discovery rate (FDR) threshold. Canonical variables (features) with significant associations (above the green line) were used for drug response modeling.

FIGS. 11A and 11B show scIDUC predictions in the presence of potential bath effects. FIG. 11A: Rho statistics comparing predicted nAUCs between I-BET sensitive cells and resistant cells over seven drugs. FIG. 11B: Cohen's D comparing predicted nAUCs between I-BET sensitive cells and resistant cells over seven drugs.

### DETAILED DESCRIPTION

Described herein is a versatile method that infers cell-level drug response in scRNA-seq data facilitates development of therapeutic strategies to target heterogeneous subpopulations within a tumor and address issues like treatment failure and resistance.

To fill the current gap in computational tools and to establish an adaptable virtual SC drug screen platform tailored toward clinically meaningful predictions, described herein is a single-cell Integration and Drug Utility Computation also referred to herein as “scIDUC”. scIDUC is a novel and transparent transfer learning based framework that quickly and accurately generates predictions of drug responses for scRNA-seq data. scIDUC learns relationships between drug sensitivities and relevant gene expression patterns based on CCL RNA-seq data and CCL high-throughput drug screens. Integration of CCL gene expression and target scRNA-seq datasets is performed to denoise and extract shared gene expression patterns between bulk and SC data sources; the resulted bulk component is then used to train drug response models, whose coefficients are further applied to post-integration SC data to infer cellular drug sensitivity scores. The method is evaluated using a variety of scRNA-seq datasets with known cellular drug sensitivity status. Moreover, through prospective analysis in three distinct scenarios, the versatility of the framework is demonstrated in in various biological models addressing research questions and generating meaningful therapeutic predictions with potential clinical impact. Validation of predictions yielded from scIDUC substantiates its potential as a first-line research tool in the field of computational drug discovery for addressing intratumoral heterogeneity and to facilitate hypothesis formulation for various oncology research topics.

scIDUC learns relationships between drug sensitivities and relevant gene expression patterns based on CCL RNA-seq data and CCL drug screen results from databases such as the Cancer Therapeutics Response Portal Version 2 (CTRPv2) (Rees et al., 2016, Seashore-Ludlow et al., 2015, Basu et al., 2013) and the Cancer Cell Line Encyclopedia (CCLE) (Barretina et al., 2012). Integration of CCL RNA-seq dataset and target scRNA-seq dataset is performed to de-noise and extract shared gene expression patterns between bulk and single cell (SC) data sources; the resulted bulk data is then used to train drug response models, whose coefficients are further applied to SC data to infer cellular drug sensitivity scores.

The method is evaluated using a variety of scRNA-seq datasets with known cellular drug resistance status. Through prospective analysis in three distinct scenarios, it is further demonstrated the versatility of the framework described herein in various biological models addressing research questions and generating meaningful therapeutic predictions with potential clinical impact. Evaluation of predictions yielded from scIDUC substantiates its potential as a first-line study tool in the field of computational drug discovery for addressing intratumoral heterogeneity and to facilitate hypothesis formulation for various oncology research topics.

To generate drug response scores for each cell, scRNA-seq data is integrated with current cancer cell line bulk RNA-seq data to obtain shared gene expression patterns. Canonical correlation analysis is employed to capture similarities between cells from scRNA-seq data and bulk RNA-seq data. Feature selection is conducted to extract drug response informative features. This enables re-mapping of the original data onto a “subspace” with significantly lower dimensionality and less noise to facilitate downstream predictions. Known drug-gene relationships are applied to scRNA-seq data to predict cellular drug response in a transfer learning-based approach. Due to its scalability, capability of handling large data sets, and ability to integrate scRNA-seq data with multiple drug screen databases, the technology and methods described herein demonstrate higher accuracy in predicting drug response scores compared to existing methods such as Beyondcell and CaDRReS-Sc for example. This technology may support the development of targeted therapies and the creation of more personalized treatment plans for heterogeneous cancers.

While the technology is described herein with respect to examples carried out with respect to cancer drug development as the scIDUC may support cancer drug development by enabling efficient investigations of intra-tumor/tissue therapeutic vulnerability, the application of the scIDUC technology is not so limited. Additional applications include use as a clinical decision-making tool where scIDUC may support clinical decision making by informing combination drug predictions to create more personalized patient treatments for heterogeneous cancers. Further applications include use as a research tool where scIDUC technology may be an effective research tool for scientists studying drug responses in single-cell RNA sequencing data, enabling deeper insights into cellular behavior and drug sensitivity.

Assessment of gene expression at the single cell level offers detailed mappings of cell compositions and substantially advances understanding of complex diseases, including but not limited to cancer, that involves heterogeneous cell types. Origins of therapy resistance and disease recurrence have been linked to such heterogeneity in many malignancies, often attributed to existence of insusceptible cells thriving under selective pressures (Jamal-Hanjani et al., 2015, Schmidt et al. 2016, McGranhan et al. 2017). Accordingly, cell-type-aware drug discovery using scRNA-seq data has demonstrated potentials to curb resistance and improve treatment outcomes (Sade-Feldman et al. 2018, Cohen et al., 2021, Abdelfattah et al. 2022). Though computational drug discovery tools have been proposed for this goal amid increasing public access to scRNA-seq datasets, most pipelines still focus on target identification and validation, a procedure often involves generation of scRNA-seq data, or inference of cellular changes under therapeutic perturbations (instead of cellular response to a potential treatment given its expression profile) (Filipp, 2019, Van de Sande et al., 2023). These methods ultimately fall short at utilizing existing scRNA-seq data to conduct virtual drug screens, facilitating hypothesis formulation, and proposing drug candidates addressing tumor heterogeneity. scIDUC as discussed herein fills this niche. scIDUC comprises a novel computational pipeline that generates predictions of drug responses for scRNA-seq data.

ScIDUC first integrates the pan-cancer CCL bulk RNA-seq and scRNA-seq data based on CCL-SC similarities. By coercing both datasets to have the same DRGs, we obtain embeddings that preserve the similarities between bulk and scRNA-seq data in a drug response relevant context, allowing predictions to be made at the single cell level without the need for scRNA-seq training data, which is difficult to acquire. Based on high-throughput drug screen databases such as the CTRPv2 and the GDSC (Rees et al., 2016, Seashore-Ludlow et al., 2015, Basu et al., 2013, Yang et al., 2013), scIDUC predicts cellular vulnerabilities to various drugs and has shown considerable ability to detect resistant and sensitive cell populations. More importantly, through prospective analysis and validation in three distinct scenarios, we demonstrated the versatility of scIDUC for quickly generating cell-type-specific predictions to address resistance and heterogeneity given scRNA-seq data. The resulting predictions showed high concordance with previous findings and experiment results, further bolstering the utility of scIDUC for providing therapeutic opportunities with potential clinical impact.

A few other methods have been proposed to computationally predict single cell drug response across cancer therapeutics such as Beyondcell, CaDRRes-sc, and scDEAL. Each employ a transfer learning based approach, utilizing relationships between bulk expression data with known drug response to predict single cell level drug sensitivity. Beyondcell infers an enrichment score from bulk data, used to identify therapeutic clusters within the scRNA-seq data and guide drug selection. CaDRRes-sc employs supervised based approaches with matrix factorization to learn a latent pharmacogenomic space. scDEAL utilizes an autoencoder based pipeline. However, the current implementation of scDEAL only supports pre-built single-cell data and faces reproducibility issues. Comparing scIDUC according to one or more embodiments herein with CaDRRes-sc and Beyondcell demonstrated scIDUC's superiority in simplicity, interpretability, and accuracy. It is also noteworthy that compared with other methods, scIDUC requires minimal parameter tuning, enabling adaptations to a broad user base for various oncology therapy research topics.

Successful characterization of drug response profiles at the single cell level plays a fundamental role in advancing precision medicine in cancers (Filipp, 2019, Qi et al., 2023). Learned cellular sensitivity to various drugs can greatly benefit studies tackling topics such as heterogeneity and cancer drug resistance by providing cell type specific therapy vulnerability information. Aided by the robust prediction results from scIDUC, the present disclosure relates to drug candidate identification in diverse biological scenarios including PDXs (RMS-oPDX), TME (PDAC-CFPAC1), and in vitro cell lines (CRPC-CCLs). Drugs predicted to have preferential efficacy against cell groups of interest were prioritized; the proposed candidates achieved high consistency compared with initial findings from corresponding publications and in vitro experiment results. These validated prospective analyses demonstrated the utility of scIDUC to quickly enable well-founded theories for downstream investigations. Serving as an alternative to traditional drug panels and target identification, scIDUC was able to provide valuable therapy development options without the hassle involved in experiments.

In addition, knowledge of intratumoral therapy vulnerability has been explored to inform formulation of drug combinations that target multiple cell groups to help eliminate heterogeneous tumors (Wu et al., Dagogo et al., 2018). Given the vast number of potential combination therapies, computational frameworks have been proposed to conduct virtual systematic screens for specific indications (Ling et al. 2011, Kong et al., 2022). To this end, cellular drug response scores are key components for modeling combination efficacies. For example, nAUC might be perceived as the probability of an organism surviving a certain drug treatment; under such an assumption, cellular nAUCs can be used to infer potential drug synergy under the null hypothesis and principles of independent drug action (where drugs do not interact (Plana et al., 2022). Cellular drug response scores from scIDUC provide key variables for drug combination modeling strategies; scIDUC and computational drug combination discovery pipelines can be used to establish a virtual screen platform for various complex cancers.

### Example Method

### Data Acquisition

Pan-cancer cell line (CCL) transcriptomic data (bulk RNA-seq) was downloaded from the Cancer Dependency Map (DepMap, https://depmap.org/portal/) (Basu et al., 2013) and originally from the Cancer Cell Line Encyclopedia (CCLE) (Barretina et al., 2012). CCLE expression data was downloaded in count and log 2(TMP+1) formats. CCL drug response data was downloaded from DepMap, originally from the Cancer Therapeutics Response Portal (CTRPv2) generated at the Broad Institute (Rees et al., 2013, Seashore-Ludlow et al., 2015, Basu et al., 2013). Embodiments described herein utilize raw drug screen data to refit dose-response curves and retain robust drug sensitivity profiles (Simplicity v1.0, 2023). For each drug-CCL pair, area-under-the-dose-response-curve (AUC) was divided by its tested dose range to generate normalized AUC (nAUC), which was then used conjointly with CCL bulk RNA-seq in scIDUC; nAUC is continuous and ranges between 0 to 1 with 0 implying complete cell kill and 1 implying no cell kill.

The single-cell RNA-seq datasets used were downloaded from various sources, depending on the availability of original data provided by the authors. A detailed description of each data source can be found in TABLE 1, below.

TABLE 1
scRNA-seq datasets

| | | No. | | | | |
|---|---|---|---|---|---|---|
| Data | | of | Sample | | Drug | |
| Name | Authors | Cells | Source | Disease | Name | Availability |
| Lung- | Kong et | 507 | PC9 cell | Lung cancer | Gefitinib | GSE112274 |
| PC9 | al. | | line | | | |
| AML- | Bell et al. | 1472 | MLL- | Acute Myeloid | I-BET-151 | GSE110894 |
| PDX | | | AF9 | Leukemia | | |
| Breast- | Ben- | 2899 | MCF7 | Breast cancer | Bortezomib | GSE114462 |
| MCF7 | David et | | cell line | | | |
| | al.^{a} | | | | | |
| RMS- | Patel et | 5643 | Patient- | Rhabdomyosarcoma | SN-38 and | NA^{b} |
| oPDX | al. | | derived | (RMS) | EGFRis | |
| | | | xenografts | | | |
| CRPC- | Schnepp | 324 | PC3 and | Castration-resistant | Docetaxel | GSE140440 |
| CCLs | et al. | | DU145 | prostate cancer | | |
| | | | cell lines | (CRPC) | | |
| PDAC- | Raghavan | 2042 | CFPAC1 | Pancreatic ductal | SN-38 and | Single Cell |
| CFPAC1 | et al. | | cell line | adenocarcinoma | Paclitaxel | Portal |
| | | | | (PDAC) | | #1644^{c} |

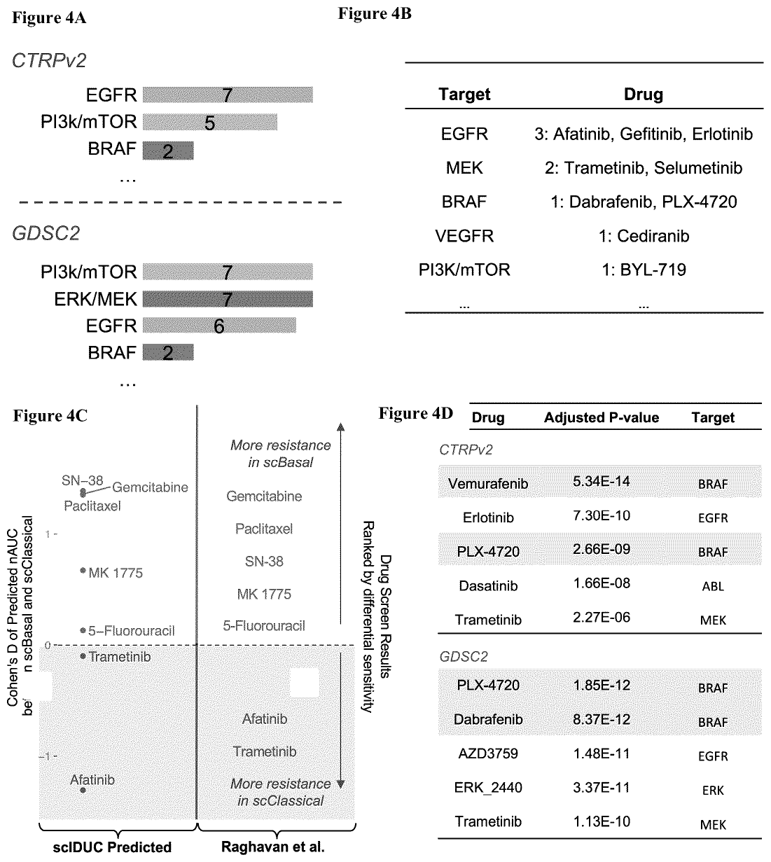

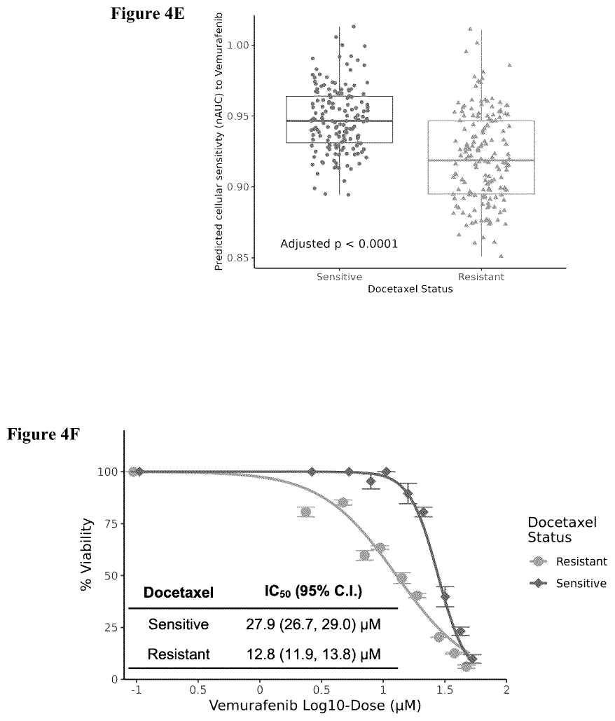

^{a}t0 cells treated as Bortezomib sensitive cells and t96 cells as resistant cells.
^{b}The RMS data in our study was obtained from Dr. Anand G. Patel, originally generated at the St. Jude Children's Research Hospital.
^{c}CFPAC1 cell line data presented in FIG. 4 in Raghavan et al. (2021) was used.

### Preprocessing

CCL names in downloaded CCLE transcriptome and CTRPv2 drug response data were harmonized to Cellosaurus accession numbers, which make use of the prefix “CVCL” (Ling et al., 2018, Bairoch 2018). Given that a drug was only screened in a subset of CCLs, percentages of missing values for each drug were calculated and the method included excluding those screened in less than 40 percent of all CCLs in the database. This results in a total of 493 treatments (and 887 CCLs) in the CTRPv2 dataset.

For scRNA-seq data with raw counts, each dataset was pre-processed using the Scanpy Python module (McGraw et al., 1922). A universal threshold was imposed on all datasets to filter for cells with at least 200 genes detected and genes detected in at least 3 cells. Each cell was then normalized to have the same total counts of 1 million (counts per million, CPM) and log-transformed through log 2(CPM+1).

### Drug Response Relevant Genes Generation

The R package limma was used to detect drug response relevant genes (DRGs) given the continuous nature of nAUC. For each drug, CCLE gene expression counts were used to construct linear models. Resulting genes were ranked by B-statistics which indicates probabilities of differentially expressed from most significantly associated with drug response to least.

### Integration of Bulk and Sc Data

Two different approaches were implemented to integrate bulk and single-cell RNA-seq data, namely canonical correlation analysis (CCA) and non-negative matrix factorization (NMF). Where X_{bk} ∈ [symbol] ^{n1xp} is the CCL bulk RNA-seq matrix with n₁ samples and p genes; X_{sc} ∈ R^{n2xP} is a scRNA-seq matrix with n₂ cells and p genes. The bulk- and sc-matrices have the same DRGs (p).

### Integration via CCA

The CCA integration pipeline proposed in the Seurat package (Stuart 2019) was expanded. A singular value decomposition (SVD) on the matrix derived based on the multiplication of X_{bk}X_{sc}^{T}. X_{bk}X_{sc}^{T}. captures similarities between CCLs from bulk RNA-seq and cells from scRNA-seq based on the shared DRGs. Therefore, resulting singular vectors through SVD, i.e.,

$$SVD ( {X}_{bk} {X}_{sc}^{T} ) = {USV}^{T} = {\sum }_{i = 1}^{k} {u}_{i} \cdot {s}_{i} {v}_{i}^{T} \qquad ( 1 )$$

can be viewed as canonical correlation vectors (CCVs). The left singular vectors U=(u₁, u₂, . . . , uₙ_{₁}) correspond to CCVs for bulk data; the right singular vectors V=(v₁, v₂, . . . , Vₙ_{₂}) correspond to CCVs for single-cell data.

### Accordingly,

$${Z}_{bk} = U \sqrt{S} ∈ {ℝ}^{{n}_{1} \times k} , \qquad ( 2 )$$

$$and$$

$${Z}_{sc} = V \sqrt{S} ∈ {ℝ}^{{n}_{2} \times k} \qquad ( 3 )$$

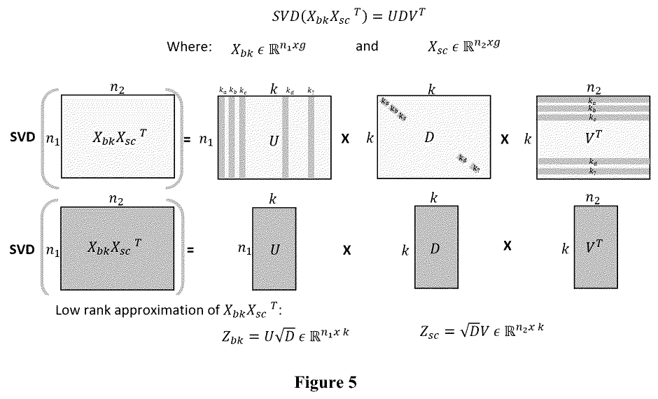

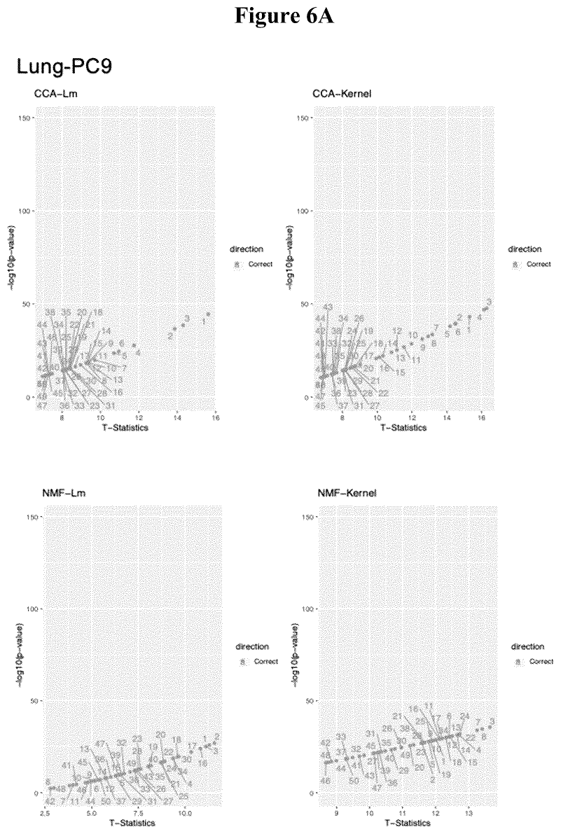

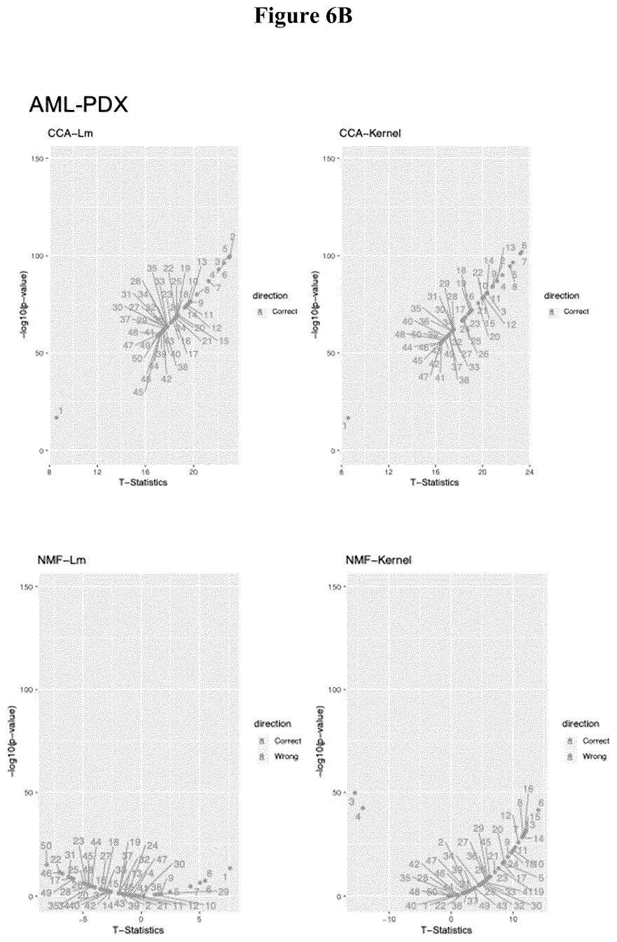

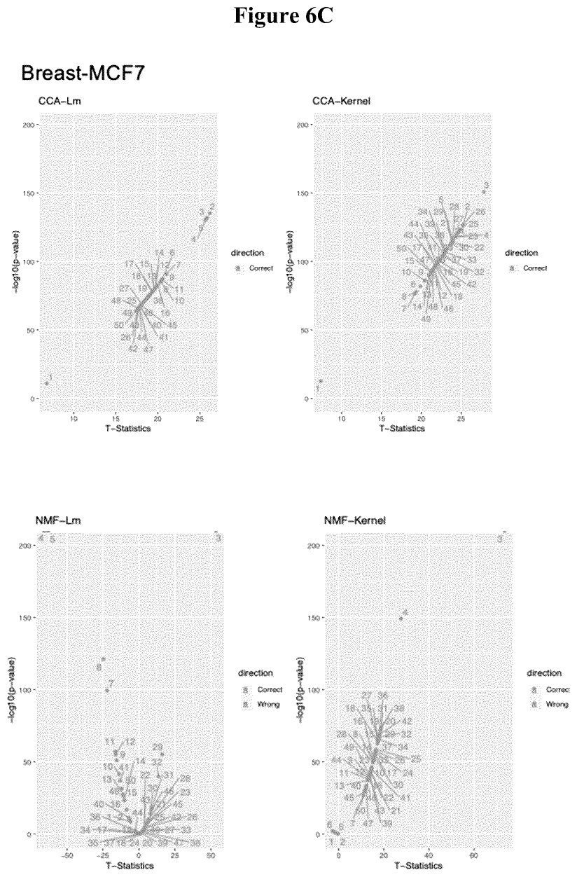

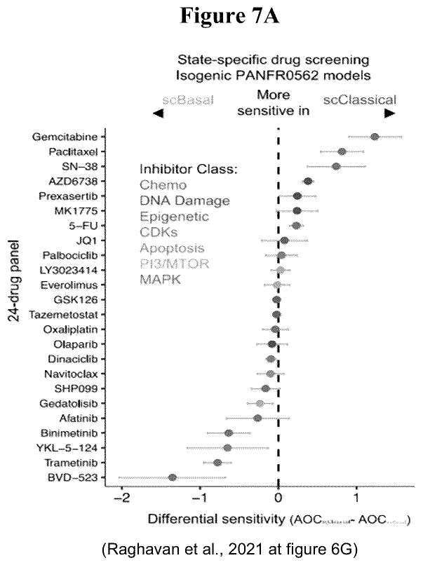

provides embeddings of X_{bk} and X_{sc} to a subscape where similarities between bulk and single-cell data are preserved. A visualization of this process is provided in FIG. 5.

### Integration via NMF

Since several studies have reported NMF-based methods to capture common gene expression patterns and adjust for discrepancies between SC batches (Liu et al., 2020, Peng et al., 2021), embodiments herein included NMF as an alternative means to integrate bulk and single-cell data. With a concentrated matrix containing both data sources defined as:

$$Y = [ {X}_{bk}^{T} , {X}_{sc}^{T} ] ∈ {ℝ}^{p \times ( {n}_{1} + {n}_{2} )} , \qquad ( 4 )$$

$$then$$

$$NMF ( Y ) = WH \qquad ( 5 )$$

where W ∈ R^{kxn1} is a common factor matrix whose columns can be viewed as “metagenes”; H ∈ R^{kx(n1+n2)} describes metagene expression profiles for bulk samples and single cells. Within H, H_{bk} ∈ R^{kxn1} and H_{sc} ∈ R^{kxn2} are metagene expression matrices for bulk data and single-cell data, respectively.

### Drug-Response Relevant Feature Extraction

Ad hoc feature extraction is performed to select a pharmacogenomic subspace (CCA) or pharmacogenomic metagenes (NMF) for accurately inferring single-cell drug response. For embeddings resulted from CCA integration, embodiments correlate each dimension (feature) in Z_{bk} (bulk embeddings) with measured drug response. Resulting p-values are adjusted via the Benjamini-Hochberg procedure to control false discovery rates (FDRs) (Benjamini et al., 1995). Dimensions that have FDRs less than a threshold δ are retained. In other words, such pharmacogenomic subspace comprises dimensions r={r₁, r₂, . . . , rⱼ}⊏{1, 2, . . . , k}where FDR(rⱼ)<δ and δ ∈ {0.05, 0.1} by default. Training data is then defined as:

$${X}_{train} = {Z}_{bk}^{{n}_{1} \times r} , \qquad ( 6 )$$

predictions of cellular drug response will be made on

$${X}_{test} = {Z}_{sc}^{{n}_{2} \times r} . \qquad ( 7 )$$

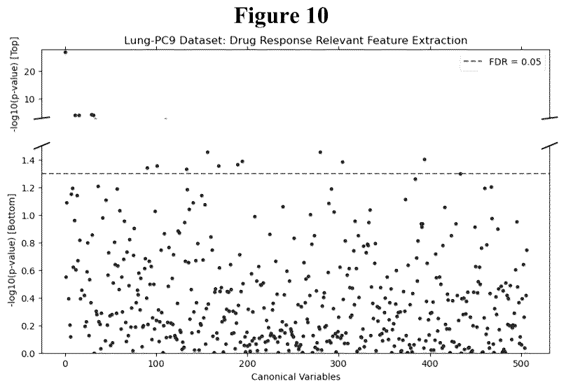

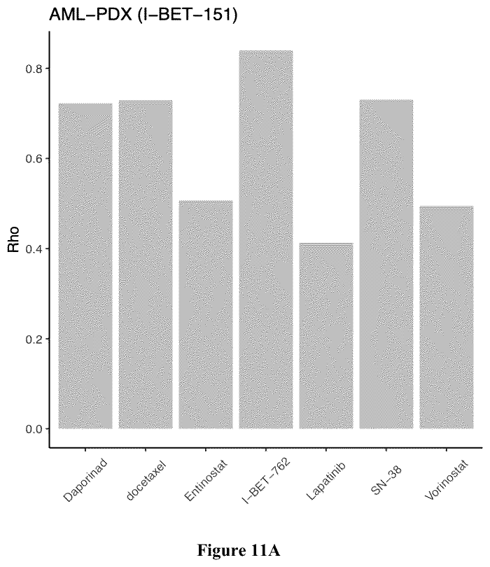

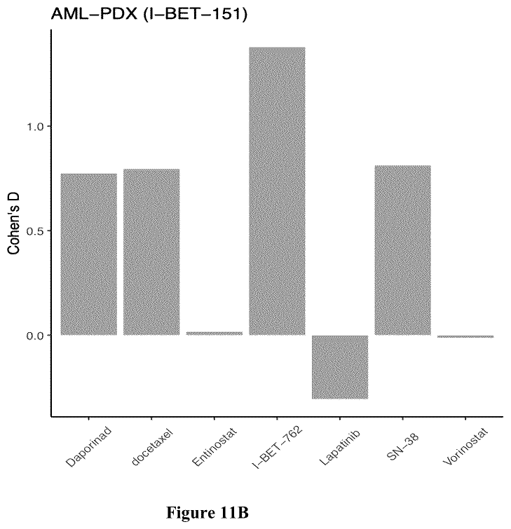

A Manhattan plot showing each feature's correlation coefficient p-value using the Lung-PC9 dataset is shown in (FIG. 10).

For NMF, megagenes were selected by correlating each metagene in H_{bk} with measured drug response. Metagenes that have FDRs less than a threshold w are kept. Embodiments including retaining a set of metagenes m={m₁, m₂, . . . , mᵢ}⊏{1, 2, . . . , k}, where FDR(mᵢ)<π. Training data is then defined as:

$${X}_{train} = {( {H}_{bk}^{m \times {n}_{1}} )}^{T} , \qquad ( 8 )$$

predictions of cellular drug response will be made on

$${X}_{test} = {( {H}_{sc}^{m \times {n}_{2}} )}^{T} . \qquad ( 9 )$$

### SC Drug Response Prediction

To predict single-cell drug response, embodiments herein include formulating a regression model using Xₜᵣₐᵢₙ as predictors and measured drug response as the dependent variable. Learned coefficients are then applied to Xₜₑₛₜ to generate nAUCs for cells. An alternative non-parametric regression model based upon the radial basis function (RBF) kernel can also be included.

### Evaluation Metrics

To evaluate the performances of algorithms, embodiments comprise comparing predicted cellular nAUCs against true drug sensitivity status (resistant or sensitive) via two-sample t-tests. To better illustrate, two additional metrics showing the effect sizes of predicted drug response differences between resistant and sensitive cell groups may be incorporated.

Letting the predicted nAUCs of resistant cells be Lᵣ=(lᵣ₁, lᵣ₂, . . . , l_{rP}) and that of sensitive cells be Lₛ=(lₛ₁, lₛ₂, . . . , l_{sq}), the common language effect size (p) is a non-parametric statistic describing the probability that a randomly selected cell from Lᵣ will have a greater nAUC than a randomly sampled cell from the Lₛ(McGraw et al., 1992). Thus, p can be directly calculated via the Mann-Whitney U-statistic:

$$ρ = \frac{U}{p \times q} \qquad ( 10 )$$

This is also equivalent to the area-under-the-receiver-operating-characteristic-curve (AUC-ROC). Therefore a larger p indicates a more accurate prediction result.

We also calculate Cohen's D as a parametric effect size which provides a measure of robustness and variation in addition to differences between two cell groups:

$$Cohen ’ s D = \frac{{L}_{r}^{-} - {L}_{s}^{-}}{s} \qquad ( 11 )$$

where s is the pooled standard deviation from the two groups, i.e.,

$$s = \sqrt{\frac{( p - 1 ) {s}_{{L}_{r}}^{2} + ( q - 1 ) {s}_{{L}_{s}}^{2}}{p + q - 2}} . \qquad ( 12 )$$

A value of 0.8 or higher is typically viewed as a large effect size and above (Lakens, 2013).

### Cellular Drug Response Prediction Via CaDDReS-Sc and Beyondcell

CaDDReS-Sc by default supports GDSC drug screen. While the CaDRReS-Sc github repository suggests flexibility to train a new model based on other drug response datasets like CTRP, this pipeline was not applied to CTRP in neither the original manuscript nor github. To allow for comparison between other similar methods, CTRPv2 was used. To calculate the weight for each sample-drug pair that is determined by the logistic weight function, max concentrations and AUC as the original pipeline learned weights from max concentrations and IC50s can be used. To generate predictions on the single cell (SC) samples, the kernel features as the correlations between CCLs and SC samples can be defined. Embodiments eliminated application of the Bayesian sigmoid curve fitting approach to raw intensity data, recomputing IC50 values and thus did not apply this method to AUC values. To generate predictions on the single cell (SC) samples, embodiments described herein can be defined the kernel features as the correlations between GDSC cancer cell lines and single cell samples. For Beyondcell, embodiments may utilize gene signatures generated from only the CTRPv2 database to infer cellular drug sensitivity scores (Beyondcell Scores or BCS, TABLE 1). Since a higher BCS indicates higher sensitivity, comparing sensitive cells against resistant cells may ensure consistent directions with the rest of the methods. One crucial parameter when deriving BCS is the expression threshold by which cells with low detected expression levels are penalized and dropped. A grid search conducted with each scRNA-seq dataset to determine an expression threshold that propagated optimal results while retaining at least 90 percent of original cells can be utilized. Bootstrap aggregation was performed, and performance was summarized across 50 applications of scIDUC and CaDRReS-Sc where 95% of bulk samples were randomly selected for each application. Given that Beyondcell provides pre-trained signatures, embodiments sampled 95% of up- and down-regulated genes without replacement as a bootstrap experiment. See TABLE 3, below.

TABLE 3
Benchmarking scIDUC against other competing methods*

| | | | -LOG10(P- | |
|---|---|---|---|---|
| Data | Method | Cohen's D | value) | Rho |
| PDAC_Paclitaxel | Beyondcell | 0.55 (0) | 33.37 (0) | 0.65 (0) |
| PDAC_Paclitaxel | CaDRReS-Sc | 0.39 (0.01) | 17.36 (0.71) | 0.39 (0) |
| PDAC_Paclitaxel | scIDUC-CCA | 1.22 (0.25) | 143.98 (44.6) | 0.8 (0.05) |
| Schnepp | Beyondcell | 0.94 (0.02) | 14.32 (0.6) | 0.74 (0) |
| Schnepp | CaDRReS-Sc | 0.29 (0.02) | 2.01 (0.17) | 0.57 (0) |
| Schnepp | scIDUC-CCA | 1.78 (0.12) | 42.11 (4.07) | 0.89 |
| | | | | (0.02) |
| SJRHB013758_X1 | Beyondcell | 0.94 (0.02) | 14.32 (0.6) | 0.74 (0) |
| SJRHB013758_X1 | CaDRReS-Sc | 0.12 (0.01) | 2.66 (0.21) | 0.47 (0) |
| SJRHB013758_X1 | scIDUC-CCA | 2.07 (0.16) | Inf | 0.93 |
| | | | | (0.01) |

*The values are presented as mean (SD)

### Cell Culture and Reagents

A DU145 prostate cancer cell line was obtained from American Type Culture Center (ATCC) and cultured in RPMI 1640 medium (Thermo Fisher Scientific), supplemented with 10% fetal bovine serum (FBS) (Gibco, Thermo Fisher Scientific) and maintained at 37° C. with 5% CO₂. A docetaxel-resistant cell line model for DU145 was established by chronically exposing the parent cell line to stepwise increasing concentrations of docetaxel as previously described(41,42). Both cell lines were periodically monitored for mycoplasma using the Universal Mycoplasma Detection Kit following the manufacturer's protocol (A TCC). In vitro drug screening in both the docetaxei-resistant and control DU145 nodels was performed using either vemurafenib (PLX4032; CAS No. 918504-65-1) or docetaxel (RP-56976; CAS No. 114977-28-5) obtained from MedChem Express (Monmriouth Junction NJ, USA) dissolved in dimethylsulfoxide (DMSO) to obtain stock concentrations of 100 mM for vemurafenib or 5 mM for docetaxel.

### Drug Screening in Docetaxel-Resistant and Control DU145 Cell Lines

Docetaxel-resistant and control DU145 cells were trypsinized, harvested, counterstained with Hoechst 33342 Fluorescent Stain (Thermo Scientific, Pierce Biotechnology, Rockford, IL) and resuspended in full growth media to 5×10⁴ cells per mL prior to plating in 96-well microplates (Thermo Scientific) using a seeding density of 5×10³ cells per well and allowed to attach for 24 hours. Following incubation, cells were treated with different concentrations of either docetaxel ranging from 0.92 nM to 6 uM, or vemurafenib ranging from 2.5 uM to 50 uM. Cell viability for each well was measured following a 72-hour drug exposure using the WST-1 assay [(Roche Applied. Science, Penzberg, Upper Bavaria, Germany) following the manufacturer's protocol. Absorbance at the 450 nm wavelength was assessed using the Synergy HTX Multi-Mode Plate Reader (BioTek, Winooski, VT)]. Absorbance values for each well were used to calculate percent viability relative to the no drug condition. Results are reported as a mean and standard deviation of three independent biological experiments, each containing three technical replicates for each experimental condition.

Heterogeneous cell groups within tumors often show varying sensitivities to oncology treatments and have been recognized as a pivotal factor for therapeutic resistance and disease progression. Through single-cell sequencing techniques, characterization of molecular backgrounds at the cellular level has become increasingly available, which calls for specialized approaches for translating current understanding of intratumoral heterogeneity into new treatment strategies to advance patient care. One key step in development of such approaches centers around accurate prediction of drug response at the single-cell level. One or more embodiments described herein utilize a novel computational framework that integrates pan-cancer cell line and target single-cell transcriptomic profiles and predicts therapeutic efficacies for the target single-cell sequencing data. Embodiments described herein illustrate that the approaches described herein achieve high accuracy in projecting cellular drug response in various biological models. More importantly, methods described herein enable drug discovery with potential clinical impact, highlighting its trailblazing property in aiding rapid hypothesis generation in many oncology research topics.

### Framework Overview

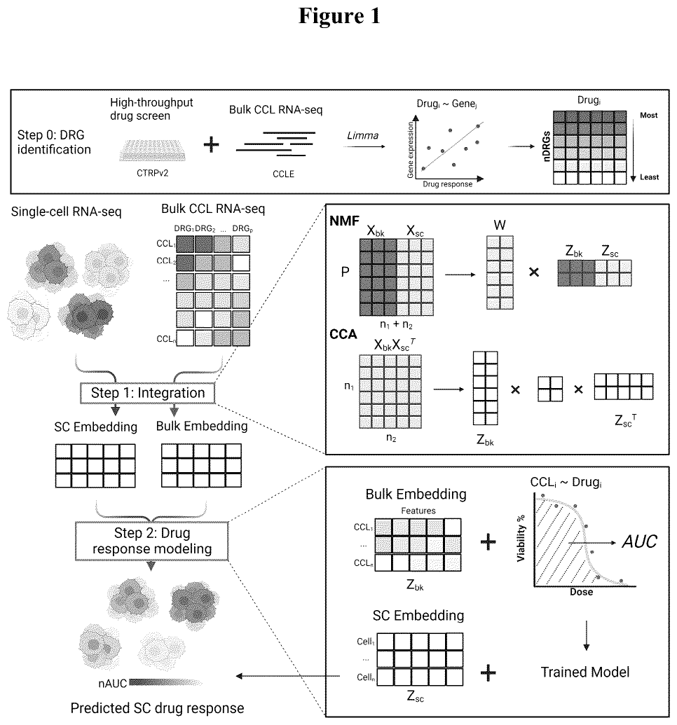

An overview of one embodiment of the scIDUC technology including a computational pipeline is outlined in FIG. 1. Drug screen results from the CTRPv2 in normalized area-under-the-dose-response-curve (nAUC) were used as CCL drug response. Prior to main computation steps, identification of drug response relevant genes (DRGs) for each drug was carried out (Step 0). Instead of filtering for a certain number of DRGs using an arbitrary threshold, a typical DRG list for a drug consists of all genes that are ranked from most drug response informative to the least. This is to partially address that different drug may display different predictability in different scRNA-seq data. The input bulk and SC RNA-seq datasets were then subset to retain the same DRGs.

Given the distinct properties between bulk RNA-seq and scRNA-seq data due to technological differences, scIDUC first integrates the two data sources to preserve shared gene expression patterns and to parse out less relevant noise (Step 1). In this step, imposing DRGs maximizes the likelihood of retaining shared transcriptomic patterns that are associated with drug response. Also, given different input scRNA-seq data might have varying quality, using DRGs may be more resilient than including all genes indiscriminately for predicting drug response. Many data integration methods have been proposed to merge multiple scRNA-seq datasets. The scRNA-seq analysis R package Seurat has incorporated canonical correlation analysis (CCA) as one of the core algorithms to combine multiple SC datasets, based on the rationale that CCA will preserve similarities between data sources (Stuart et al., 2019). Also, non-negative matrix factorization (NMF) has been used for joining scRNA-seq datasets, partially given the interpretation of its inner decomposition factors as “metagenes” (Liu et al., 2020, Peng et al., 2021). In Step 1, embodiments described herein examine and/or extrapolate both CCA and NMF algorithms for integrating bulk and SC datasets, which without being bound by theory contain less commonalities compared with merging SC datasets only. Embodiments are also designed to further evaluate performances of CCA and NMF for accurate drug response predictions. Some embodiments The integration step generates embeddings of the two input RNA-seq datasets and projects them into a low dimensional space. Next in Step 2 regression-based approach is utilized to model drug response. Resulting embeddings of CCL RNA-seq are used as predictors and measured drug response as the response. Coefficients of the subspace features were then applied to SC embeddings to infer cellular drug response. This yields predicted nAUC values for all cells in the scRNA-seq data as inferred response to drugs in the CTRPv2.

### Selection of Parameters and Evaluation of Pipeline Performances

Based on the overall design of scIDUC, one crucial parameter is the number of DRGs, as it directly affects data integration and drug response performances. Since the input bulk RNA-seq data mostly remains constant (CCL expression profile) while the target scRNA-seq varies, determining this parameter in a data-dependent way, codifying this parameter as the ratio between number of SCs and number of DRGs (or SC-DRG ratio) was achieved. In addition, the flexible pipeline also allows for evaluation of different means of integration (Step 1) and drug response modeling (Step 2).

Thus, to establish an optimal structure of the pipeline described herein, scIDUC is applied with different parameters or settings to three independent scRNA-seq datasets with known sensitivity status to specific drugs. To be broadly applicable, datasets were chosen that represent various diseases and biological origins. In these data, drug resistance was established through chronic exposing model system(s) to drugs of interest, followed by scRNA-seq of both parent drug-sensitive and derived drug-resistant model(s). Specifically, in the Lung-PC9 dataset, Kong et al. chronically exposed PC9 lung cancer cells to gefitinib (Kong et al., 2019) to establish resistance. In the second dataset (Breast-MCF7)(Ben-David et al., 2018), Ben-David et al. developed Bortezomib resistant cells derived from the MCF7 breast cancer cell line. Bell et al. generated cells resistant to BET inhibitors from murine acute myeloid leukemia patient derived xenografts (AML-PDX) models (TABLE 1)(Fong et al., 2015, Bell et al., 2019).

The predicted cellular drug response from scIDUC was compared with the true sensitive/resistant labels. Two experiments investigating impacts from (1) means of data integration and drug response modeling as well as from (2) the SC-DRG ratio parameter were conducted.

Predicted cellular drug sensitivities from scIDUC were evaluated via calculating two effect sizes between the true resistant and sensitive groups, namely the common-language effect size (Rho statistic) and the Cohen's D effect size. R Rho statistics indicate the probability of a randomly selected cell from the true resistant group having higher predicted nAUC than a randomly selected cell from the true sensitive group (see Methods). A value less than 0.5 indicates contradictory predicted drug response status, a value around 0.5 implies random chance, and a value above 0.5 is ideal. Similarly, Cohen's D describes differences in predicted nAUCs between the two groups while considering variability (see Methods). A Cohen's D can generally be interpreted as having a small effect size at 0.2, a medium effect size at 0.5, and a large effect size at 0.8(40). Higher values of either criterion therefore signify better performances.

Embodiments described herein applied scIDUC with varying parameter settings on these dataset (as described previously above) and compared predicted cellular drug response with the true sensitive/resistant labels.

(1) Selection of integration methods (CCA or NMF) and drug response modeling strategies

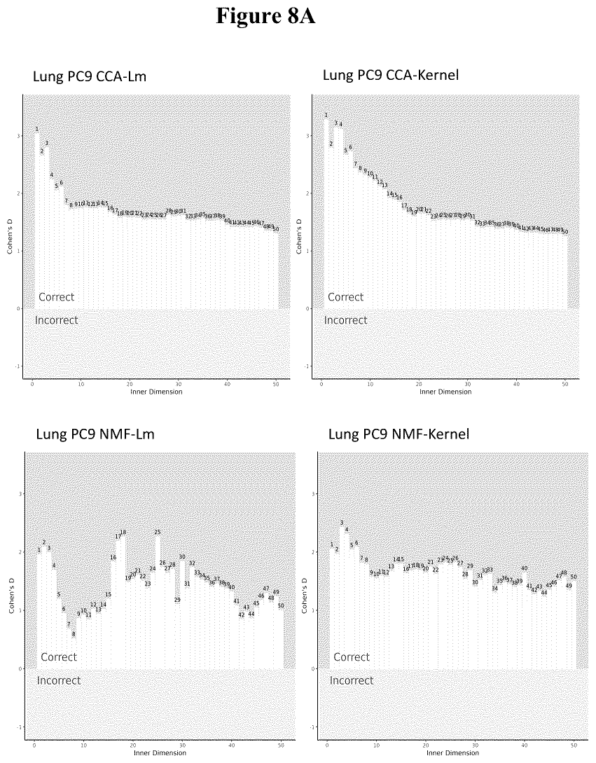

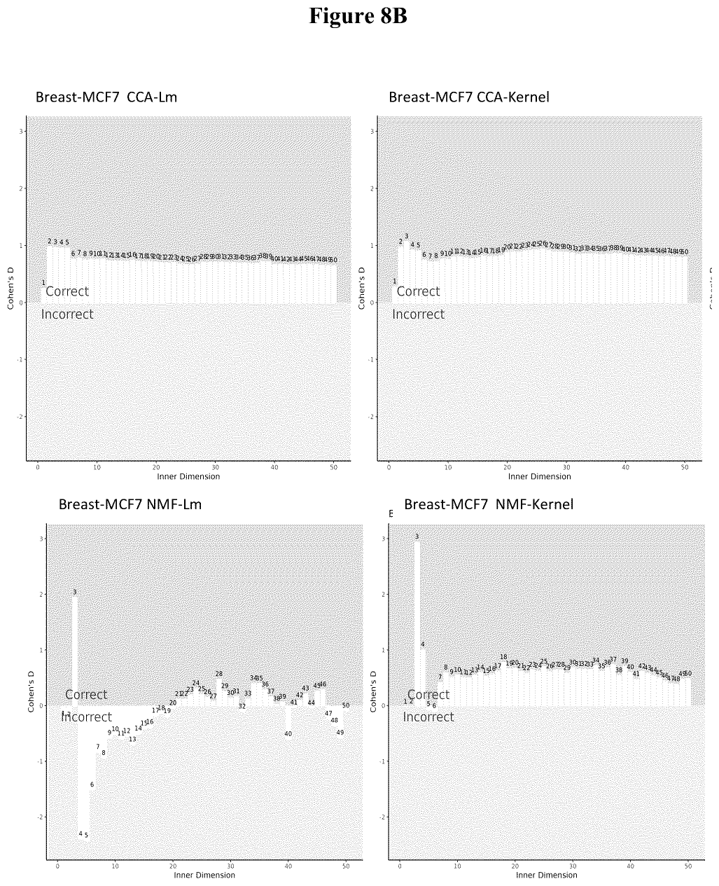

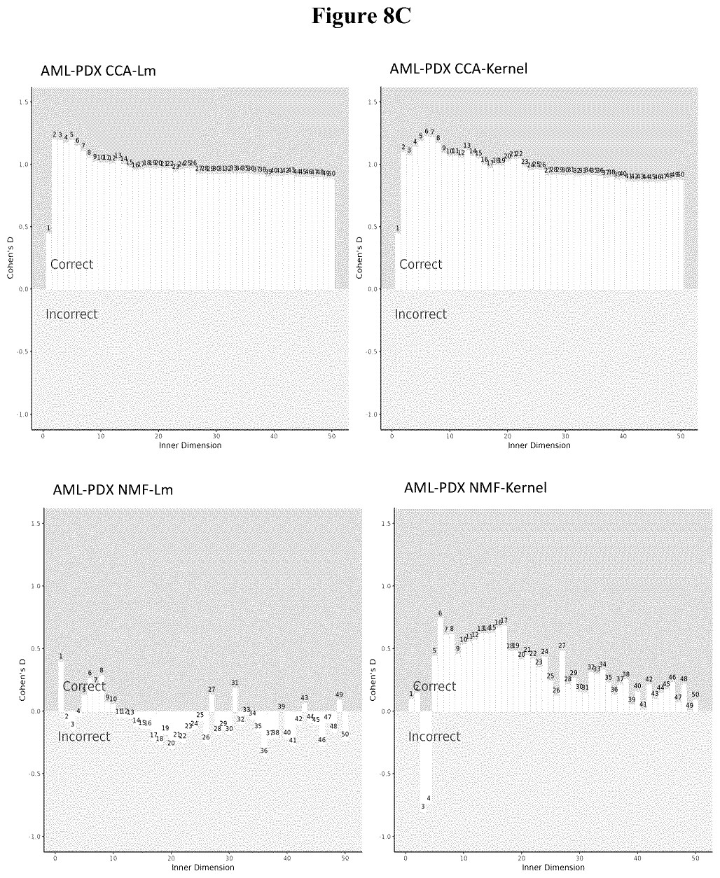

Different formulae in both data integration (Step 1) as well as in modeling drug response (Step 2) were probed. Given that one crucial parameter for both methods is the inner dimension k (number of latent factors for NMF and number of canonical correlation vectors, or CCVs, for CCA), extensive investigations into the robustness of each integration approach with varying k values (k={1,2, . . . ,50}) were carried out. Drug response modeling incorporated linear regression (Lm) and non-parametric regression models based on a Gaussian kernel (Kernel) to ascertain the optimal pipeline. The first k CCVs (for CCA) and the first k latent factors (or metagenes, for NMF) respectively, and examined predicted single-cell drug sensitivities against the truth were examined by reporting Cohen's D (FIG. 8). For Lung-PC9, both methods were able to generate correct sensitivity trends towards gefitinib, while CCA based integration shows superiority in Cohen's D. For Breast-MCF7 and AML-PDX, CCA consistently predicted correct cellular drug response status, whereas volatile results were observed with NMF, especially NMF with linear models for drug response prediction. Although NMF coupled with kernelized regression resulted in more stable results than NMF and linear models, CCA continued to show superior results regardless of regression models. Taken together, CCA integration showed superiority over NMF regarding both accuracy and robustness. When coupled with CCA, both regression models gave comparable results. Additionally, while the selection of the optimal k in either CCA or NMF plays a central role in algorithm performance given that the computational goal is to model drug response, we implemented feature selection on post-integration embeddings to include subspace features that correlate with drug response (see above and FIG. 10). Through this, scIDUC was able to quickly select only a few informative features for model training and prediction without screening for optimal inner dimensions in an unsupervised fashion.

### (2) Identification of Optimal SC-DRG Ratios:

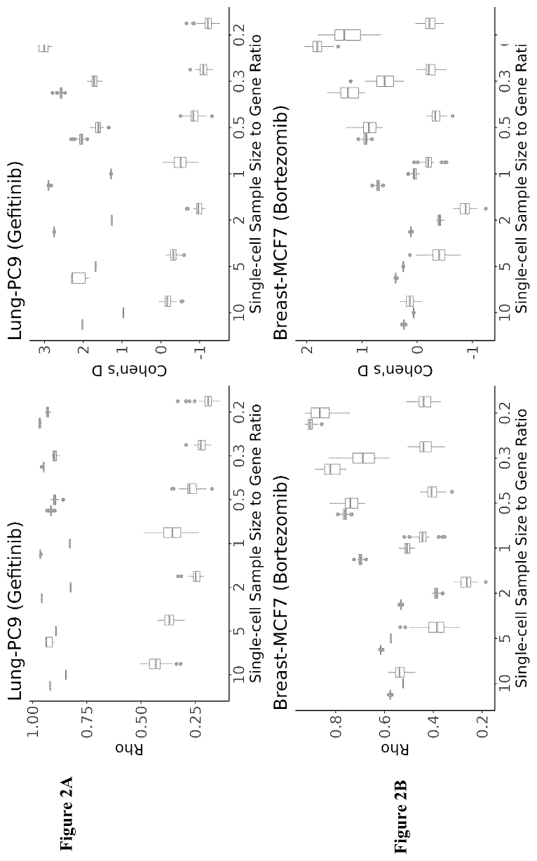

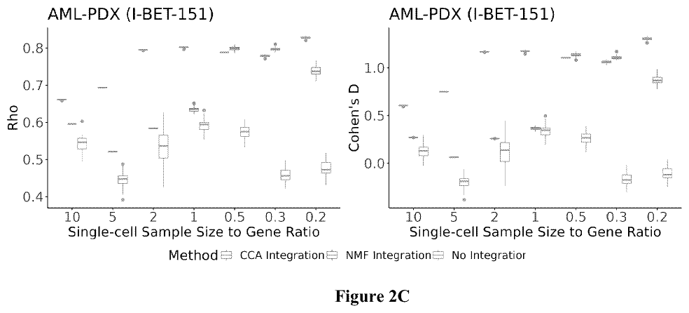

Given results from (1), further testing of varying SC-DRG ratios when using (a) no integration, (b) CCA+Lm, and (c) NMF+Kernel occurred. An SC-DRG ratio range spanning from 10 to 0.2 was examined. For each SC-DRG, in Step 2, a subset of 95% of available CCLs were randomly selected as a bootstrap sample to train prediction models. This process is repeated 50 times, which allowed us to test scIDUC's stability and robustness. Evaluation results were reported for each SC-DRG ratio (FIG. 2). When integration was performed, median Cohen's D surpassed 0.8 and median Rho statistics surpassed 0.5 for the majority of ratios tested, implying that scIDUC can in general recapitulate known cellular response to drugs across three scRNA-seq datasets regardless of SC-DRG ratios. No clear trend was observed from results without integration. Predicted nAUCs with integration also showed higher robustness, indicated by less variability in the results. While with both integration algorithms the prediction accuracy tends to peak when SC-DRG ratios fell between 1 and 0.2, NMF showed higher variability compared to CCA and had poorer performances outside the ideal SC-DRG ratio range (FIGS. 2 and 10). A SC-DRG ratio between 1 to 0.2 in general gave good results, supported by stable Rho statistics close to 1 and Cohen's D larger than 1 across all three scRNA-seq data.

Taking results from (1) and (2) together, prioritizes CCA as scIDUC's core integration method. Linear regression is employed to model drug response for its simplicity and interpretability. A SC-DRG ratio between 0.2 and 1 is recommended for obtaining optimal predictions. However, the final build of scIDUC package does allow users to explore NMF and kernel regression as alternative settings.

TABLE 2
scIDUC performance with different integration algorithms and different cell-to-DRG ratios.

| | | | Cells to DRGs Ratios^{a} | | | | | | | | | |
|---|---|---|---|---|---|---|---|---|---|---|---|---|
| Data | Method | Metric | 10 | | 5 | | 2 | | 1 | | 0.5 | |
| Bell | CCA | −LOG10(P- | 14.91 | (13.46) | 23.53 | (19.15) | 50.22 | (45.57) | 53.39 | (43.23) | 46.69 | (40.08) |
| | Integration | Value) | | | | | | | | | | |
| Bell | CCA | Cohen's D | 0.61 | (0) | 0.75 | (0) | 1.17 | (0) | 1.18 | (0) | 1.11 | (0) |
| | Integration | | | | | | | | | | | |
| Bell | CCA | Rho | 0.66 | (0) | 0.69 | (0) | 0.8 | (0) | 0.8 | (0) | 0.79 | (0) |
| | Integration | | | | | | | | | | | |
| Bell | NMF | −LOG10(P- | 0.55 | (0) | 1.6 | (0) | 0.33 | (0) | 0.96 | (1.34) | 4.58 | (4.72) |
| | Integration | Value) | | | | | | | | | | |
| Bell | NMF | Cohen's D | 0.27 | (0) | 0.06 | (0) | 0.26 | (0) | 0.37 | (0.02) | 1.14 | (0.02) |
| | Integration | | | | | | | | | | | |
| Bell | NMF | Rho | 0.6 | (0) | 0.52 | (0) | 0.58 | (0) | 0.64 | (0.01) | 0.8 | (0) |
| | Integration | | | | | | | | | | | |
| Bell | No Integration | −LOG10(P- | 14.91 | (13.46) | 23.53 | (19.15) | 50.22 | (45.57) | 53.39 | (43.23) | 46.69 | (40.08) |
| | | Value) | | | | | | | | | | |
| Bell | No Integration | Cohen's D | 0.1 | (0.05) | −0.21 | (0.06) | 0.15 | (0.19) | 0.34 | (0.06) | 0.27 | (0.06) |
| Bell | No Integration | Rho | 0.54 | (0.02) | 0.44 | (0.02) | 0.54 | (0.05) | 0.59 | (0.02) | 0.57 | (0.02) |
| Ben- | CCA | −LOG10(P- | 6.98 | (3.75) | 26.78 | (15.94) | 55 | (56.14) | 41.5 | (33.58) | 72.17 | (54.36) |
| David | Integration | Value) | | | | | | | | | | |
| Ben- | CCA | Cohen's D | 0.24 | (0.01) | 0.39 | (0.01) | 0.11 | (0.01) | 0.71 | (0.04) | 0.93 | (0.05) |
| David | Integration | | | | | | | | | | | |
| Ben- | CCA | Rho | 0.58 | (0) | 0.62 | (0) | 0.53 | (0) | 0.7 | (0.01) | 0.76 | (0.01) |
| David | Integration | | | | | | | | | | | |
| Ben- | NMF | −LOG10(P- | 3.33 | (0) | 0.28 | (0) | 9.61 | (12.26) | 5.99 | (6.41) | 12.65 | (13.8) |
| David | Integration | Value) | | | | | | | | | | |
| Ben- | NMF | Cohen's D | 0.06 | (0) | 0.25 | (0) | −0.41 | (0.04) | 0.05 | (0.05) | 0.88 | (0.16) |
| David | Integration | | | | | | | | | | | |
| Ben- | NMF | Rho | 0.52 | (0) | 0.57 | (0) | 0.39 | (0.01) | 0.51 | (0.02) | 0.74 | (0.04) |
| David | Integration | | | | | | | | | | | |
| Ben- | No Integration | −LOG10(P- | 6.98 | (3.75) | 26.78 | (15.94) | 55 | (56.14) | 41.5 | (33.58) | 72.17 | (54.36) |
| David | | Value) | | | | | | | | | | |
| Ben- | No Integration | Cohen's D | 0.13 | (0.09) | −0.4 | (0.2) | −0.87 | (0.12) | −0.21 | (0.1) | −0.34 | (0.1) |
| David | | | | | | | | | | | | |
| Ben- | No Integration | Rho | 0.54 | (0.02) | 0.39 | (0.05) | 0.27 | (0.03) | 0.44 | (0.03) | 0.41 | (0.03) |
| David | | | | | | | | | | | | |
| Kong | CCA | −LOG10(P- | 13.58 | (12.84) | 14.62 | (13.19) | 22.71 | (14.62) | 21.9 | (18.97) | 14.26 | (8.64) |
| | Integration | Value) | | | | | | | | | | |
| Kong | CCA | Cohen's D | 2.02 | (0) | 2.15 | (0.18) | 2.75 | (0) | 2.89 | (0.01) | 2.06 | (0.09) |
| | Integration | | | | | | | | | | | |
| Kong | CCA | Rho | 0.92 | (0) | 0.93 | (0.01) | 0.96 | (0) | 0.96 | (0) | 0.91 | (0.01) |
| | Integration | | | | | | | | | | | |
| Kong | NMF | −LOG 10(P- | 1.68 | (0) | 2.92 | (0) | 24.71 | (0) | 3.88 | (0) | 0.81 | (0.89) |
| | Integration | Value) | | | | | | | | | | |
| Kong | NMF | Cohen's D | 0.96 | (0) | 1.68 | (0) | 1.26 | (0) | 1.28 | (0) | 1.61 | (0.09) |
| | Integration | | | | | | | | | | | |
| Kong | NMF | Rho | 0.85 | (0) | 0.89 | (0) | 0.82 | (0) | 0.83 | (0) | 0.9 | (0.01) |
| | Integration | | | | | | | | | | | |
| Kong | No Integration | −LOG10(P- | 13.58 | (12.84) | 14.62 | (13.19) | 22.71 | (14.62) | 21.9 | (18.97) | 14.26 | (8.64) |
| | | Value) | | | | | | | | | | |
| Kong | No Integration | Cohen's D | −0.19 | (0.13) | −0.34 | (0.1) | −0.97 | (0.1) | −0.52 | (0.22) | −0.86 | (0.16) |
| Kong | No Integration | Rho | 0.43 | (0.04) | 0.37 | (0.03) | 0.25 | (0.02) | 0.36 | (0.06) | 0.27 | (0.04) |

| | Cells to DRGs Ratios^{a} | | | | | |
|---|---|---|---|---|---|---|
| Data | 0.3 | | 0.2 | | All | |
| Bell | 41.82 | (39.36) | 58.45 | (56.99) | 70.52 | (70.06) |
| Bell | 1.06 | (0.01) | 1.31 | (0.01) | 1.48 | (0.03) |
| Bell | 0.78 | (0) | 0.83 | (0) | 0.86 | (0.01) |
| Bell | 6.93 | (7.77) | 6.72 | (5.96) | 26.76 | (13.57) |
| Bell | 1.11 | (0.02) | 0.87 | (0.05) | 0.48 | (0.05) |
| Bell | 0.8 | (0) | 0.74 | (0.01) | 0.62 | (0.01) |
| Bell | 41.82 | (39.36) | 58.45 | (56.99) | 70.52 | (70.06) |
| Bell | −0.15 | (0.06) | −0.1 | (0.08) | 0.01 | (0.07) |
| Bell | 0.46 | (0.02) | 0.48 | (0.02) | 0.51 | (0.02) |
| Ben- | 106.57 | (103.98) | 50.39 | (100.46) | 125.5 | (138.82) |
| David | | | | | | |
| Ben- | 1.24 | (0.2) | 1.79 | (0.14) | 1.59 | (0.1) |
| David | | | | | | |
| Ben- | 0.82 | (0.04) | 0.9 | (0.02) | 0.88 | (0.01) |
| David | | | | | | |
| Ben- | 11.72 | (13.09) | 16.16 | (19.22) | 5.99 | (8.02) |
| David | | | | | | |
| Ben- | 0.58 | (0.21) | 1.27 | (0.31) | 0.15 | (0.11) |
| David | | | | | | |
| Ben- | 0.68 | (0.06) | 0.86 | (0.05) | 0.55 | (0.05) |
| David | | | | | | |
| Ben- | 106.57 | (103.98) | 50.39 | (100.46) | 125.5 | (138.82) |
| David | | | | | | |
| Ben- | −0.23 | (0.12) | −0.23 | (0.12) | −0.28 | (0.1) |
| David | | | | | | |
| Ben- | 0.43 | (0.03) | 0.44 | (0.03) | 0.42 | (0.03) |
| David | | | | | | |
| Kong | 22.27 | (11.38) | 28.71 | (14.94) | 8.07 | (5.65) |
| Kong | 2.58 | (0.05) | 3.01 | (0.11) | 1.45 | (0.08) |
| Kong | 0.95 | (0) | 0.97 | (0) | 0.85 | (0.01) |
| Kong | 0.96 | (1.03) | 0.74 | (0.59) | 19.23 | (7.41) |
| Kong | 1.71 | (0.1) | 2.01 | (0.08) | 1.2 | (0.2) |
| Kong | 0.9 | (0.01) | 0.93 | (0.01) | 0.85 | (0.05) |
| Kong | 22.27 | (11.38) | 28.71 | (14.94) | 8.07 | (5.65) |
| Kong | −1.09 | (0.12) | −1.2 | (0.16) | −0.44 | (0.2) |
| Kong | 0.22 | (0.02) | 0.2 | (0.03) | 0.39 | (0.05) |

^{a}values are presented as mean (SD).

### Outcomes

scIDUC Outperforms Other Methods in scRNA-Seq Data from Various Sources

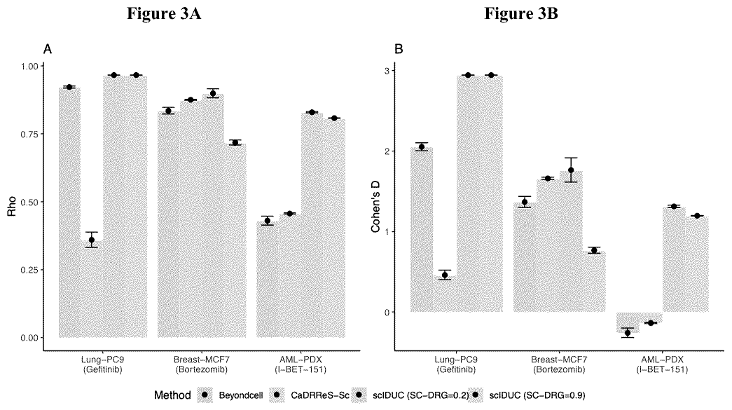

scIDUC was benchmarked against other methods, namely Beyondcell (Fuster-Torre 2021) and CaDRReS-Sc (Suphavilai et al., 2021), which also aim to predict cellular drug response using additional scRNA-seq data, representing various biology backgrounds (TABLE 1). In CRPC-CCLs, Schnepp et al. exposed castration-resistant prostate cancer (CRPC) cell lines PC3 and DU145 to docetaxel to acquire resistance (Schnepp 2020). In RMS-oPDX, Patel et al. discovered a mesoderm-like cell colony using scRNA-seq on orthotopic patient-derived xenografts (oPDX) from pediatric patients with rhabdomyosarcoma (RMS). These cells were shown to be highly resistant to the chemotherapy irinotecan (whose active metabolite is SN-38) compared to myoblast cells but sensitive to EGFR inhibitors (Patel et al., 2022). The PDAC-CFPAC1 dataset describes ductal pancreatic adenocarcinoma CFPAC1 cells whose drug response was profoundly altered by tumor microenvironment (TME) (Raghavan et al., 2021). When growing in complete classical organoid media, CFPAC1 cells lost basal properties and became responsive to several treatments including SN-38 and paclitaxel. This data allowed investigation of performances of scIDUC and competing methods in diverse biology models and indications. Benchmarked prediction performances of scIDUC, Beyondcell, and CaDRReS-Sc used the same evaluation criteria, namely Cohen's D and Rho statistics. Bootstraping was implemented for all three methods. For scIDUC, since a SC-DRG range between 0.2 to 1 generally produced good results based on our experiment results from FIG. 2, using two SC-DRG ratios (0.2 and 0.9) within this range demonstrates the minimal parameter tuning needs for scIDUC, while avoiding biased results. Across all datasets, the scIDUC method according to embodiments described herein achieves the highest effect sizes across all three benchmarking datasets, with median Rho-statistics above 0.8 (FIG. 3A) and median Cohen's D above 1 (FIG. 3B). Beyondcell was able to separate the true resistant and sensitive cells and showed high consistency, however its results were less accurate (median Rho-statistics around 0.7 and median Cohen's D less than 1) compared with scIDUC. CaDRReS-Sc did not generate meaningful predictions to recollect cellular drug response (median Rho-statistics around 0.5 and median Cohen's D close to 0).

Additionally, across datasets Lung-PC9, Breast-MCF7, and AML-PDX, similar results were observed: scIDUC in general had the highest Rho statistics and Cohen's D; Beyondcell provided meaningful predictions though less accurate. Interestingly, CaDRReS-Sc showed good performance with the Breast-MCF7 dataset, though failed to recapitulate true cellular drug response status in the other two. Notably, for AML-PDX, neither Beyondcell nor CaDRReS-Sc was able to recollect true cell drug response status (FIGS. 3A, 3B). The suboptimal performances of the two other methods are not unanticipated given their methodological designs. For Beyondcell, though applying bulk-learned signatures to SC data could bypass integrating bulk and SC datasets and to some extent reflect cellular response to drugs, these signatures rely on quality of scRNA-seq data. Signature score calculation can be subordinate to random factors such as drop-outs and low expression values, resulting in less ideal predictions. CaDRReS-Sc centers its modeling strategies around IC50 instead of AUC, which has been demonstrated to be more reliable for response prediction (Kurilov R., et al. 2020). Here, the instant benchmarking results could suggest that modeling strategies used by CaDRReS-Sc lack adaptivity to account for AUCs as drug response. Furthermore, a fixed set of drug response essential genes were used by CaDRReS-Sc for all drugs. Since different drugs might correlate with different genes, an invariant gene set might be insufficient to capture drug-gene relationships. In comparison, scIDUC and Beyondcell are both sensitive to drug-specific genes and showed superior results. scIDUC enables clinically meaningful drug discovery

To showcase the utility of the scIDUC computational framework described herein as a trailblazer in addressing research questions and aiding hypothesis development, prospective analyses for three different scenarios utilizing the RMS-oPDX, PDAC-CFPAC1, and CRPC-CCLs datasets, representing diverse biology models including patient-derived xenografts (PDX), tumor microenvironments (TME), and acquired drug resistance in vitro were conducted.

In RMS-oPDX, scIDUC was applied to screen efficacious drugs targeting the SOC (SN-38) resistant mesoderm-like cells. The nominated drugs showed high consistency with findings from the original study. In PDAC-CFPAC1, scIDUC was used to predict sensitivities of CFPAC1 cells grown in different TMEs to various drugs. The resulting drug differential efficacy profile between the two TMEs were highly comparable to drug panel results reported by Raghavan et al. Finally, in CRPC-CCLs, scIDUC was utilized to screen drugs showing efficacies in docetaxel-resistant CRPC cells and successfully validated our nomination through in vitro experiments.

### Nominating Drugs Targeting Mesoderm Cells in RMS Patients

In RMS-oPDX, Patel et al. delineated that mesoderm-like cells in pediatric RMS tumors bore profound resistance to SOC chemotherapy irinotecan (SN-38) (Patel et al., 2022). Therefore, targeting therapy-resistant mesoderm cells constitutes a cornerstone for curbing the current high rates of disease recurrence. To this end, embodiments herein include application of scIDUC to RMS-oPDX, aiming to discover drugs showing high efficacy in mesoderm cells. To increase likelihood of finding actionable therapeutics, the pool of candidate drugs was expanded by predicting cellular sensitivities to various compounds from the CTRPv2 as well as the Genomics of Drug Sensitivity in Cancer 2 (GDSC2) (Yang et al., 2013) databases. Two-sample testing was performed between mesoderm cells and myoblast cells, through which drugs displaying lower predicted nAUC (suggesting higher sensitivity) in mesoderm cells were included as candidates. To ensure prediction robustness, this pipeline was applied independently to oPDX from each patient (11 in total) in the dataset, and frequency of each nominated drug was summarized over all patients. Embodiments herein considered drugs with a frequency higher than 50%, or nominated at least in 6 patients independently as robust candidates. Embodiments identified drugs with diverse mechanisms and ranked their target pathways by the number of drugs belonging to the same class (FIG. 4A). In both CTRPv2 and GDSC2, epidermal growth factor receptor (EGFR) is among the most frequent targets (FIG. 4A). Furthermore, 16 drugs were simultaneously proposed to be efficacious against mesoderm cells by comparing prediction results from both databases. The top five target pathways of the 16 identified compounds are presented in FIG. 4B, in which EGFR is ranked the first, targeted by three drugs (Afatinib, Gefitinib, and Erlotinib). Drug nomination according to the methods described herein is strongly supported by the original study, where EGFR was validated as an actionable drug targeted for chemo-resistant mesoderm cells. The application of scIDUC recapitulates such a finding through independent, data-driven analysis. In addition, scIDUC has proposed other drugs and target pathways as potential strategies for inhibiting mesoderm cells in RMS patients, such as MEK inhibitors (Trametinib and Selumetinib).

### Depicting Therapeutic Susceptibility in PDAC Cells Affected by Different TMEs

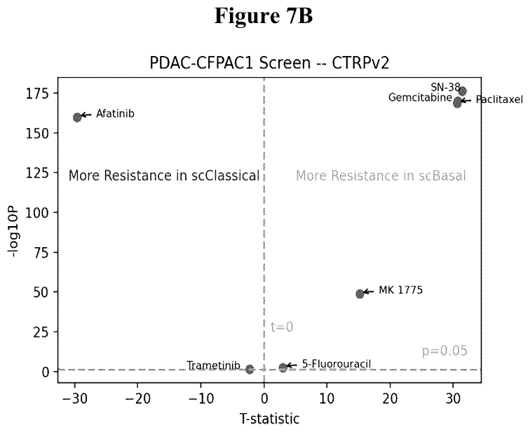

Raghavan et al. showed that TME dramatically altered sensitivities to various therapeutics in the PDAC cell line model CFPAC1 as well as in PDAC organoids (Raghavan et al., 2021). For example, as previously recapitulated by scIDUC, CFPAC1 cells grown in classical complete organoid media (scClassical) were re-sensitized to SN-38 and Paclitaxel compared to their counterparts grown in the basal cell line media (scBasal). An additional drug screen panel was performed by the authors to obtain a broader differential drug response profile between scBasal and scClassical states induced by different TMEs (FIGS. 7A, 7B). To study predictability of scIDUC in this situation, embodiments include predicting sensitivities of scBasal and scClassical cells to the same treatments tested in the original drug panel. Based on the drug panel, treatments with demonstrated differential efficacy (i.e., treatments whose mean differential efficacy deviated from zero and confidence interval did not contain zero) were kept, among which seven drugs were found in the CTRPv2 database and used by scIDUC to generate cellular response predictions. Two-sample t-tests were conducted to examine differences in predicted nAUCs between scBasal and scClassical cells (FIGS. 7A, 7B). Cohen's D was calculated to demonstrate differences in predicted drug response between scBasal and scClassical cells; a positive Cohen's D indicates more resistance in scBasal cells, whereas a negative value implies the contrary. A plot of Cohen's D of each treatment by its actual value to illustrate the extent of predicted differential efficacy between the two cell states is shown in FIG. 4C (Left). Corresponding drug panel results from Raghavan et al. were shown in their original ranking for comparison are also shown in FIG. 4C (Right). The instant results achieved high consistency with drug panel data by Raghavan et al. Gemcitabine, SN-38, and Paclitaxel had the highest Cohen's D, indicating profound discrepancies between resistant scBasal cells and sensitive scClassical cells. MK 1775 and 5-Fluorouracil showed modest and minimal differences, whereas Tremetinib and Afatinib were predicted to be more efficacious in scClassical cells. The scIDUC results are strongly supported by the original drug panel, which highlights the capability of scIDUC at capturing TME-shaped drug response at the single-cell level. Furthermore, scIDUC can be applied to hundreds of drugs to decipher potential differential drug response in different TMEs, which is extremely expensive if not impossible to carry out at this scale experimentally.

### Discovering and Validating Drugs for Docetaxel-Resistance in CRPC

Though docetaxel was approved for CRPC, resistance among patients is prevalent (Schnepp et al., 2020), highlighting a need to identify new therapeutics targeting the non-responsive cells. scIDUC was employed to predict cellular sensitivities to all available treatments in the CTRPv2 and the GDSC2 databases and prospectively identified efficacious drugs for docetaxel resistant cells. Two-sample t-tests were conducted comparing docetaxel resistant and sensitive cell groups, through which drugs showing higher effects (lower nAUCs) in the resistant group using an adjusted p-value threshold of less than 0.05 were selected. Resulting drugs from each database were grouped and summarized based on their known targets as illustrated in FIG. 4D. A number of BRAF inhibitors were identified from the CTRPv2 (vemurafenib and PLX-4720) and the GDSC2 (PLX-4720 and dabrafenib). In CTRPv2, vemurafenib showed the highest differential efficacy between the two CRPC cell groups, with docetaxel resistant cells showing higher predicted vulnerability (FIG. 4E, adjusted p-value=5.34×10⁻¹⁴).

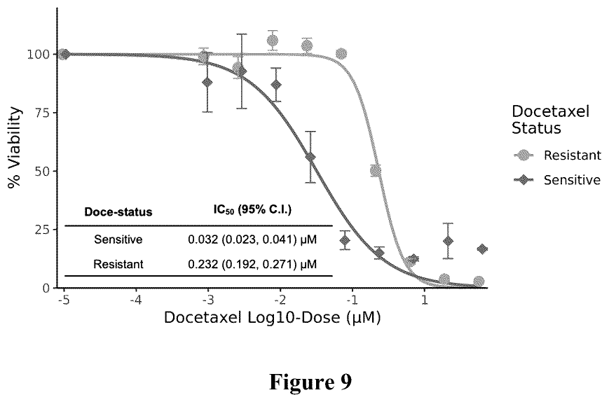

To experimentally validate this prediction, vemurafenib was testing using a previously developed in vitro cell line model system containing docetaxel sensitive and resistant DU145 cells. First, these cells were exposed t to docetaxel to ensure presence of resistance to the drug (FIG. 9). To evaluate the candidate drug, we treated cells with increasing concentrations of vemurafenib and generated dose-response curves for both cell groups (FIG. 4F). Vemurafenib showed significantly higher inhibitory activity among docetaxel resistant DU145 cells with a mean half maximal inhibitory concentration (IC50) of 12.8 μM/L compared to its IC50 of 27.9 μM/L in sensitive cells (FIG. 4F, two-way ANOVA p<0.0001). Taken together, the in vitro experiment results matched the computational predictions, further supporting the reliability of prospective results generated by scIDUC.

Overall, through applying scIDUC in three different scenarios fulfilling varying research needs, its ability to enable drug development targeting heterogeneous tumors is demonstrated. Further examination and experimental validation of the prediction results underscore the potential clinical impact of identified drugs. The diverse biological backgrounds in these cases, including PDX, TMEs, and in vitro cell models, support broad adaptations of scIDUC to enable clinically meaningful drug discovery. Consistent with the rationale of computational drug discovery, these results further substantiate the utility of scIDUC as the first step to generate predictions with translational potential in oncology drug discovery tasks.

Though computational drug discovery tools have been proposed for this goal amid increasing public access to scRNA-seq datasets, most pipelines still focus on target identification and validation, a procedure that often involves generation of scRNA-seq data, or inference of cellular changes under therapeutic perturbations (instead of cellular response to a potential treatment given its expression profile). These methods so far have not been demonstrated to be able to utilize existing scRNA-seq data to conduct virtual drug screens, facilitate hypothesis formulation, and propose drug candidates addressing tumor heterogeneity.

scIDUC, integrates pan-cancer CCL bulk RNA-seq and scRNA-seq data and infers cellular response to various drugs. By coercing both datasets to have the same DRGs, the CCA-based integration preserves the similarities between bulk and SC RNA-seq data in a drug response relevant context, allowing accurate predictions to be made at the single cell level without the need for scRNA-seq drug screen training data, which is difficult to acquire. More importantly, through prospective analysis and validation in three distinct scenarios, the versatility of scIDUC for quickly generating cell-type-specific predictions has been demonstrated.

To configure the scIDUC pipeline for optimal results, the performances with varying factors including SC-DRG ratios, integration methods, and drug response models have been evaluated against known cell drug response status in independent scRNA-seq datasets. Our Results spotlight an SC-DRG ratio range between 0.2 to 1, data integration via CCA, and linear regression models for accurate predictions. The framework also includes other high-throughput screens such as the GDSC2 and the Genentech Cell Line Screen Initiative (gCSI) if needed by the user. Since the scRNA-seq data used consisted of parental and resistant cell populations receiving different treatment, additional evidence shows that scIDUC predictions were not confounded by potential batch effects. First, comparing predicted sensitivities to a variety of drugs between true resistant and true sensitive cell groups in AML-PDX is shown in FIGS. 10A, 10B. Considering the established resistance against I-BET-151, predicted cellular response toward another BET inhibitor, I-BET-762, also showed significant differential sensitivity. However, predicted response to other drugs such as histone deacetylase inhibitors (entinostat and vorinostat), NAMPT inhibitor (daporinad), and EGFR/HER2 inhibitor (lapatinib) showed minimal to no separation between the two groups. Moreover, in the RMS-oPDX dataset, cells from an oPDX sample underwent sequencing altogether and therefore precluded batch effects. In this scenario, scIDUC not only recapitulated resistance to the SOC therapy SN-38 but provided drug nomination highly in line with the original findings.

The benchmarking results discussed herein support the rationale of using genes whose expression correlates with measured drug response. On the other hand, using signatures alone is susceptible to varying quality of scRNA-seq data which are known to have low detection rates. Meanwhile, calculated scores based on these signatures reflect only relative sensitivities within a scRNA-seq data and lack pharmacological meanings. Taken together, scIDUC achieves desirable results through incorporating both drug-specific features and bulk-SC integration. In addition, SCAD and scDEAL both employ DL methodologies to perform bulk-SC integration and drug response prediction. Both methods embody binarized labels as drug response and train corresponding models as classification problems. Given that drug response across CCLs is better described by spectrum values such as AUC (60), it is unclear if binary cellular drug labels can capture varying degrees of sensitivity in a heterogeneous tumor. Furthermore, insufficient evidence was given by either method to demonstrate how resulting SC drug response will benefit hypothesis generation and drug discovery.

Successful characterization of drug response profiles at the SC level plays a fundamental role in advancing precision medicine in cancers. Learned cellular sensitivity to various drugs can greatly benefit studies tackling topics such as heterogeneity and cancer drug resistance by providing cell type specific therapy vulnerability information. scIDUC can be used for various detections and predictions. For RMS-oPDX, scIDUC was applied independently in 11 oPDX samples to identify drugs showing efficacy against the SOC therapy resistant mesoderm cells. In the majority of the samples EGFR inhibitors were nominated as one of the potential drug classes, reiterating original findings from Patel et al. (Patel A G et al., 2022) Moreover, a number of other drug classes are identified as potential targets. For example, MEK inhibitors showed high occurrences combating mesoderm-like cells. Previous studies have established evidence that MEK inhibitors effectively inhibit RMS both in vitro and in vivo (Yohe M E et al., 2018, Danielle S G et al., 2023). Moreover, inhibition of the MAPK/ERK pathway by MEK inhibitors have been shown to downregulate mesodermal genes in embryonic stem cells (Kimura T et al., 2014). Given these findings, further evaluation of MEK inhibitors in RMS patients with disease recurrences is warranted.

In a second scenario, scIDUC was able to capture TME-shaped differential drug response in CFPAC 1 cells, a PDAC cell line model. Tumor microenvironmental niche factors have been shown to drive aggressive PDAC progression from a therapy-responsive “classical” state to a less differentiated “basal” state (Raghavan S et al., 2021, Shinkawa T et al., 2022). Thus, characterizing PDAC cellular drug response in different TME-driven states is a crucial first step to develop treatments to curb the current high mortality rate (five-year survival ˜9%). Differential cellular drug efficacies between scBasal and scClassical cells resulted from scIDUC accurately recapitulate drug panel testing results reported by Raghavan et al. (FIG. 4C). In addition, Shinkawa et al. also reported similar findings where basal-like, poorly progressed PDAC organoids showed higher resistance to Gemcitabine compared to classical-like organoids (Shinkawa T et al, 2022). These discoveries substantially support usage of scIDUC to streamline drug discovery under different TMEs without the need to conduct large scale drug screens.

Finally, scIDUC was utilized to screen for alternative therapeutics against docetaxel resistance in CRPC CCLs including DU145 and PC3. A top candidate, namely the BRAF inhibitor vemurafenib, showed consistent higher efficacy in docetaxel resistant DU145 cells than sensitive ones in vitro (see FIG. 4F). A previous trial of vemurafenib has reported that an average maximum serum concentration (Cmax) of 61.4 μg/mL, or equivalent to 125.3 μM/L, was well tolerated by patients (Zhang W et al., 2017). In vitro experiments exposing vemurafenib in docetaxel resistant cells estimated an IC50 of 12.9 μM/L, which sits well below the safety dose, highlighting its clinical potential to be used in combination with docetaxel to control CRPC progression. Furthermore, EGFR and MAPK pathway inhibitors have been proposed to combat docetaxel resistance by our computational pipeline (see FIG. 4D), consistent with previous studies suggesting that EGFR inhibitors mediate docetaxel resistance and MAPK pathway shows upregulation among DU145 docetaxel resistant cells (Hour T C et al., 2015, Lin J Z et al. 2018, Liu Z et al., 2015). scIDUC powered computational pipelines are able to quickly propose drug candidates with clinical impact. The method was able to pinpoint a selective collection of actionable drugs that are ready to be evaluated. Serving as an alternative to traditional drug screens and target identification, scIDUC was able to provide rationalized and streamlined drug nomination for therapeutic development. When combined with experimental testing, it can speed up development of efficacious treatments.

In addition, knowledge of intratumoral therapy vulnerability has been explored to inform formulation of drug combinations that target multiple cell groups to help eliminate heterogeneous tumors. Given the vast number of potential combination therapies, computational frameworks have been proposed to conduct virtual systematic screens for specific indications (Ling A et al., 2020, Kong W et al., 2022). To this end, cellular drug response scores are key components for modeling combination efficacies. For example, nAUC might be perceived as the probability of an organism surviving a certain drug treatment; under such an assumption, cellular nAUCs can be used to infer potential drug synergy under various statistical models (Plana D, 2022). Cellular drug response scores from scIDUC provide key variables for drug combination modeling strategies; our future work will incorporate scIDUC and computational drug combination discovery pipelines to establish a virtual screen platform for various complex cancers.

The computational method is capable of depicting single cell vulnerability to various drugs, establishing a foundation for cell-type-aware drug discovery combating the prevailing issue of treatment failure due to tumor heterogeneity. Case studies not only provide therapeutic options for various diseases but also substantiate the necessity of our proposed method in aiding efficient development of clinical meaningful treatments.

Although the present disclosure has been described with reference to preferred embodiments, workers skilled in the art will recognize that changes may be made in form and detail without departing from the spirit and scope of the disclosure.

1. A method for predicting a cellular drug response score comprising:
obtaining bulk RNA sequence data, wherein bulk RNA seq data is pan-cancer cell line transcriptomic data from one or more sources;
obtaining single cell RNA sequence data (scRNA-seq) from one or more sources;
integrating the bulk RNA sequence data and scRNA-seq data;
capturing similarities between the bulk data and the scRNA-seq data based on a shared drug response relative gene and using canonical correlation analysis;
extracting one or more drug-response relevant features and selecting a pharmacogenomic subspace for accurately predicting a single-cell drug response.

2. The method of claim 1 and further comprising detecting drug response relevant genes and ranking the drug response relevant genes by an associated adjusted p-value, ranking the drug response relevant genes from most significantly associated with a drug response to least.

3. The method of claim 1 and further comprising filtering the scRNA-seq data for cells with at least 200 genes detected and genes detected in at least 3 cells.

4. The method of claim 2 and further comprising normalizing each cell to have the same total counts of one million counts per million and log-transformed through log 2(CPM+1).

5. The method of claim 1, wherein integrating comprising the bulk RNA data having a sequence matrix according to X_{bk} ∈ [symbol] ^{n1xp} with n₁ samples and p genes and the single cell RNA [symbol] data has a sequence matrix of X_{sc} ∈ [symbol] ^{n2xp} with n₂ cells and p genes and wherein each of the matrices have a same drug response relative gene.

6. The method of claim 6 and capturing similarities between the bulk data and the single cell sequence data with a singular value decomposition on the matrix derived based on the multiplication of X_{bk}X_{sc}^{T}.X_{bk}X_{sc}^{T}.

7. The method of claim 6 and using SVD(X_{bk}X_{sc}^{T})=USV^{T}=Σᵢ₌₁^{k} uᵢsᵢvᵢ^{T} as a correlation vector wherein singular vectors U=(u₁,u₂, . . . uₙ_{₁}) correspond to a correlation vector for the bulk RNA sequence data and wherein singular vectors V=(v₁,v₂, . . . , vₙ_{₂}) correspond to a correlation vector for the scRNA-seq data.

8. The method of claim 1 wherein extracting comprises correlating each feature in Z for the bulk RNA sequence data embeddings with a known drug response.

9. The method of claim 8 and further comprising adjusting resulting p-values via a Benjamini-Hochberg procedure to control false discovery rates and retaining features that have a false discovery rate less than a selected threshold (S) are retained.

10. The method of claim 8 wherein the pharmacogenomic subspace comprises dimensions r={r₁,r₂, . . . , rⱼ}⊏{1, 2, . . . , k}wherein the threshold is δ ∈ {0.05, 0.1}.

11. The method of claim 8 and predicting the cellular drug response according to Xₜₑₛₜ=Z_{sc}^{n2xr}.

12. A method for predicting a cellular drug response comprising:
obtaining single cell RNA sequence data (scRNA-seq);
obtaining bulk RNA sequence data, wherein the bulk RNA sequence data is for a cancer cell line;
integrating the scRNA-seq data with bulk RNA sequence data and obtaining one or more shared gene expression patterns;
capturing similarities between the scRNA-seq data and the bulk RNA sequence data;
selecting one or more features for extracting drug response informative features; and
applying one or more known drug-gene relationships to the scRNA-seq data to predict a cellular drug response score.

13. The method of claim 12 and further comprising re-mapping of the bulk RNA sequence data onto a subspace with significantly lower dimensionality and less noise to facilitate downstream predictions.

14. The method of claim 12 and further comprising scaling the method to handle large data sets and integrating the scRNA-seq data with multiple drug screen databases for accuracy in predicting drug response scores.

15. The method of claim 12 wherein capturing similarities between the scRNA-seq data and the bulk RNA sequence data comprises canonical correlation analysis.

16. A method for facilitating drug discovery in various models by proposing cell-type-specific drug candidates comprising:
selecting one or more drug response relevant genes;
integrating input cancer cell line bulk RNA sequence data and single cell RNA sequence data and preserving shared expression patterns between the bulk RNA sequence data and the single cell RNA sequence data while reducing noise;
embedding the bulk RNA sequence data with known cancer cell line drug responses;
extracting drug response informative features from the resulting embeddings;
constructing one or more regression models; and
applying learned coefficients to single cell RNA sequence embeddings to predict cellular drug sensitivity scores.

17. The method of claim 16 wherein integrating comprises using varying single cell sample sizes to drug response relative gene ratios from 10 to less than 1.
